(19) United States
(12) Patent Application Publication
(10) Pub. No.: US 2023/0118252 A1
(43) Pub. Date: Apr. 20, 2023

(54) PROGNOSTIC AND TREATMENT METHODS FOR THYROID CANCER

(71) Applicant: Qualisure Diagnostics Inc., Calgary (CA)

(72) Inventors: Oliver Bathe, Calgary (CA); Farshad Farshidfar, San Mateo, CA (US); Steven Craig, Mollymook Beach (AU); Karen Kopciuk, Calgary (CA); Cynthia Stretch, Calgary (CA)

(73) Assignee: Qualisure Diagnostics Inc., Calgary, AB (CA)

(21) Appl. No.: 17/906,211

(22) PCT Filed: Apr. 1, 2021
(86) PCT No.: PCT/CA2021/050449

§ 371 Date: Sep. 13, 2022

Related U.S. Application Data
(60) Provisional application No. 63/004,852, filed on Apr. 3, 2020.

(51) Int. Cl. C12Q 1/6886; G16B 25/10; G16H 50/30

(52) U.S. Cl. CPC C12Q 1/6886; G16B 25/10; G16H 50/30; C12Q 2600/118; C12Q 2600/158

(57) ABSTRACT

Disclosed herein are methods determining the risk of recurrence of papillary thyroid cancer in a patient. The methods comprise isolating RNA from a tumor of the patient; determining the level of expression of two or more genes or gene products of a gene signature comprising: ATG14, MYO3A, ERCC5, SLC43A1, ABCC8, LTK, COPS2, CCNA2, BNIP3, FAM86C1P, GNG4, GCFC2, EEF1A2, TXNL4B, SEPSECS, ZNF215, KIF4A, EZH2, CDCA8, DISP1, SNX29P2, ATP1B1, ZNF620, HIST4H4, CENPL, GATAD1, C2orf88, WWC3, SKA3, HJURP, LOC728613, GTPBP8, RPRM, FBXO4, TICRR, AGFG2, TTK, TAFA2, MTMR14, WDR1, NEK2, RRAGA, EIF2A, REP15, NUDT15, LANCL2, NFATC2IP, GTPBP2, KHNYN, CLDN12, DNAH11, ASPHD1, REXO5, HIST2H2BF, C12orf76, MUC21, PGBD5, ABCC6P1, RHBDF1, CHAF1B, MOV10, CAB39L, FN1, DDX19B, BUB1, GPSM2, MSH5, ETV7, SUN1, GRAMD1C, LACTB2, LOC652276, EXOSC10, NUP210, ACOX3, UNC5CL, GNAO1, CGN, ZC3H18, CTSC, MFSD13A, and CCDC183; and determining the risk of PTC recurrence using the expression levels of the two or more genes.

### TECHNICAL FIELD

The present disclosure generally relates to methods for determining the risk of reoccurrence of a cancer in a patient. More specifically, the present disclosure relates to methods for determining level of risk of recurrence of papillary thyroid cancer (PTC) in a patient.

### BACKGROUND

Thyroid cancer is the 8^{th} most common cancer by prevalence, with incidence increasing by more than 6% per year since 1992. Papillary thyroid cancer (PTC) accounts for most thyroid cancers and the rising incidence of thyroid cancer can be almost entirely attributed to an increased detection rate of small PTCs. Typically, PTC has a favorable prognosis and can often be cured. However, approximately 10-15% of PTCs display a more aggressive behavior and are often resistant to conventional adjuvant therapies such as radioactive iodine. Given the increasing number of PTC cases (and the potential burden on healthcare systems), accurate prognosis is becoming increasingly important. Accurate prognosis and determination of risk of recurrence can avoid unnecessary surgery, tests, and follow-up appointments for those who receive a favourable prognosis (i.e. that there is a low-risk of PTC recurrence). Accurate prognosis and determination of risk of recurrence also means that extensive surgeries, adjuvant therapies, and prolonged follow-up appoints may be reserved for those who have aggressive PTC (i.e. a high risk of recurrence).

Currently, PTC treatment decisions are informed by the American Thyroid Association (ATA) Disease Recurrence Risk Stratification system, which estimates the risk of disease recurrence based on a number of clinical and pathological factors. However, the ATA system is unable to accurately predict recurrence of PTC. The inability of the ATA system to accurately predict the recurrence of PTC may be because the system is generally uninformed by the molecular features of the tumors. In fact, the ATA system currently only incorporates a single molecular marker, BRAF^{V600E}, when estimating the risk of disease recurrence.

As indicated above, inaccurate discrimination of PTC prognosis may result in false positives and/or false negatives in regards to aggressive PTC cases. In the case of a false positive, a patient who does not require surgery or adjuvant therapies may be administered such treatments. In addition to burdening healthcare systems, unnecessary surgeries can place needless stress on a patient's body and, in extreme cases, can cause serious or deadly injury to a patient. In the case of a false negative, a patient may not receive adequate treatment to address aggressive cases of PTC.

Thus, there remains a need for providing an accurate prognosis of PTC in order to provide patients with appropriate treatment.

### SUMMARY

The present disclosure provides methods capable of discriminating between cases of papillary thyroid cancer (PTC) having a low risk, an intermediate risk, or a high risk of recurrence in a patient by analyzing an expression pattern, or patterns, of two or more specific genes from a patient's biological sample.

Accordingly, embodiments of the present disclosure relate to methods of determining the risk of recurrence of papillary thyroid cancer in a patient, the methods comprising the steps of: (a) isolating ribonucleic acid (RNA) from a biological sample of the patient; (b) determining from the RNA, a level of expression of each of two or more genes or gene products of a gene signature of the present disclosure; and, (c) determining whether the patient has a low-risk, an intermediate-risk, or a high-risk of PTC recurrence based on the level of expression of the two or more genes of the gene signature.

The gene signature of the present disclosure comprises the following genes:

ATG14, MYO3A, ERCC5, SLC43A1, ABCC8, LTK, COPS2, CCNA2, BNIP3, FAM86C1P, GNG4, GCFC2, EEF1A2, TXNL4B, SEPSECS, ZNF215, KIF4A, EZH2, CDCA8, DISP1, SNX29P2, ATP1B1, ZNF620, HIST4H4, CENPL, GATAD1, C2orf88, WWC3, SKA3, HJURP, LOC728613, GTPBP8, RPRM, FBXO4, TICRR, AGFG2, TTK, TAFA2, MTMR14, WDR1, NEK2, RRAGA, EIF2A, REP15, NUDT15, LANCL2, NFATC2IP, GTPBP2, KHNYN, CLDN12, DNAH11, ASPHD1, REXO5, HIST2H2BF, C12orf76, MUC21, PGBD5, ABCC6P1, RHBDF1, CHAF1B, MOV10, CAB39L, FN1, DDX19B, BUB1, GPSM2, MSH5, ETV7, SUN1, GRAMD1C, LACTB2, LOC652276, EXOSC10, NUP210, ACOX3, UNC5CL, GNAO1, CGN, ZC3H18, CTSC, MFSD13A, and CCDC183.

Another embodiment of the present disclosure also relates to a method of determining a risk of recurrence of a papillary thyroid cancer (PTC) in a patient, the method comprising the steps of: (a) determining a level of expression of each of two or more genes of the gene signature from the RNA isolated from a biological sample of the patient; and, (b) determining if the patient has a low risk, an intermediate risk, or a high risk of recurrence of PTC based on the level of expression of the two or more genes of the gene signature.

Some embodiments of the present disclosure also relate to methods of treating a patient having PTC. The methods comprise the steps of: (a) determining a level of expression of each of two or more genes of the gene signature from the RNA isolated from a biological sample of the patient; (b) determining if the patient has a low risk, an intermediate risk, or a high risk of recurrence of PTC based on the level of expression of the two or more genes of the gene signature; and, (c) administering a treatment to the patient based on the determined level of risk of PTC recurrence.

Some embodiments of the present disclosure also relate to an in vitro method of determining the risk of recurrence of PTC in a patient, the method comprising the steps of: (a) isolating RNA from a biological sample of the patient; determining from the RNA a level of expression of two or more genes of the gene signature of the present disclosure; (b) and determining whether the patient has a low risk, an intermediate risk, or a high risk of PTC recurrence based on the level of expression of the two or more genes of the gene signature.

In an embodiment of the present disclosure, the biological sample may be a tumor sample that is obtained by fine-needle aspiration, a core biopsy, or from a surgical specimen. In some embodiments, the biological sample is a formalin-fixed paraffin embedded (FFPE) tumor sample or a frozen biopsy tumor sample. In some embodiments, the tumor sample is obtained by macrodissection or microdissection of a tumor. In some embodiments of the present disclosure, the tumor sample may be obtained by laser microdissection and/or pressure catapulting.

In another embodiment of the present disclosure, the step of determining of the level of gene expression comprises measuring the level of gene expression using a reverse-transcription polymerase chain reaction (RT-PCR), a complimentary deoxyribonucleic acid (cDNA) microarray, ribonucleic acid sequencing (RNAseq) or combinations thereof.

In yet another embodiment of the present disclosure, the step of determining the level of expression of the two or more genes or gene products of the gene signature comprises determining the level of expression of 5 or more genes of the gene signature. In a further embodiment, the step of determining the level of expression of the two or more genes of the gene signature comprises determining the level of expression of 7 or more genes of the gene signature. In a yet further embodiment, the step of determining the level of expression of the two or more genes of the gene signature comprises determining the level of expression of 20 to 60 genes of the gene signature.

In yet another embodiment of the present disclosure, the step of determining the level of expression of the two or more genes or gene products of the gene signature comprises determining the level of expression of at least two of: ATG14, MYO3A, ERCC5, SLC43A1, ABCC8, LTK, COPS2, CCNA2, BNIP3, FAM86C1P, GNG4, GCFC2, EEF1A2, TXNL4B, SEPSECS, ZNF215, KIF4A, EZH2, CDCA8, DISP1, SNX29P2, ATP1B1, ZNF620, HIST4H4, CENPL, GATAD1, C2orf88, WWC3, SKA3, HJURP, LOC728613, GTPBP8, RPRM, FBXO4, TICRR, AGFG2, TTK, TAFA2, MTMR14, WDR1, NEK2, RRAGA, EIF2A, and REP15; and the step of determining the patient's risk of PTC recurrence comprises determining if the patient has a high risk of PTC recurrence. In a further embodiment, if the patient is determined not to have a high risk of PTC recurrence, the method further comprises: determining the level of expression of at least two of the genes of the gene signature described herein; and determining if the patient has an intermediate risk or a low risk of PTC recurrence.

In yet another embodiment of the present disclosure, the step of determining the level of expression of the two or more genes or gene products of the gene signature comprises determining the level of expression of at least: ATG14, MYO3A, ERCC5, SLC43A1, ABCC8, LTK, COPS2, CCNA2, BNIP3, FAM86C1P, GNG4, GCFC2, EEF1A2, TXNL4B, SEPSECS, ZNF215, KIF4A, EZH2, CDCA8, DISP1, SNX29P2, ATP1B1, ZNF620, NUDT15, LANCL2, NFATC2IP, GTPBP2, ZNF215, KHNYN, CLDN12, DNAH11, EZH2, ASPHD1, REXO5, HIST2H2BF, C12orf76, MUC21, PGBD5, ABCC6P1, RHBDF1, CHAF1B, MOV10, CAB39L, FN1, DDX19B, and BUB1.

In yet another embodiment of the present disclosure, if the patient is determined to have a high risk of PTC recurrence, the treatment may comprise performing a total thyroidectomy, administering an adjuvant radioactive iodine (RAI) therapy, administering an immune checkpoint inhibitor, or a combination thereof. In another embodiment of the present disclosure, if the patient is determined to have an intermediate risk of PTC recurrence, the treatment may comprise performing active surveillance, performing a hemithyroidectomy, administering an adjuvant radioactive iodine (RAI) therapy, or a combination thereof. For patients with an intermediate risk or a high risk of PTC recurrence, in a further embodiment, the RAI therapy may comprise a pre-treatment of administering an EZH2 inhibitor. In another embodiment of the present disclosure, if the patient is determined to have a low risk of PTC recurrence, the treatment comprises active surveillance, a hemithyroidectomy, or a combination thereof. As the skilled reader will appreciate, the treatment options for patients with the low risk or intermediate risk of recurrence of PTC may change over time with advances in medicine. The skilled reader will also appreciate that the embodiments of the present disclosure may still provide value in assessing appropriate treatment options, in light of such advances in medicine, based upon the risk categorizing made possible by the embodiments of the present disclosure.

Some embodiments of the present disclosure also relate to a system for determining a risk of recurrence of a papillary thyroid cancer (PTC) in a patient, the system comprising: at least one database for storing gene expression data; at least one server computer comprising at least one processing structure functionally interconnected to the at least one database by a network, the at least one processing structure configured for: analyzing the gene expression data to determine the level of expression of each of two or more genes or gene products of a gene signature comprising: ATG14, MYO3A, ERCC5, SLC43A1, ABCC8, LTK, COPS2, CCNA2, BNIP3, FAM86C1P, GNG4, GCFC2, EEF1A2, TXNL4B, SEPSECS, ZNF215, KIF4A, EZH2, CDCA8, DISP1, SNX29P2, ATP1B1, ZNF620, HIST4H4, CENPL, GATAD1, C2orf88, WWC3, SKA3, HJURP, LOC728613, GTPBP8, RPRM, FBXO4, TICRR, AGFG2, TTK, TAFA2, MTMR14, WDR1, NEK2, RRAGA, EIF2A, REP15, NUDT15, LANCL2, NFATC2IP, GTPBP2, KHNYN, CLDN12, DNAH11, ASPHD1, REXO5, HIST2H2BF, C12orf76, MUC21, PGBD5, ABCC6P1, RHBDF1, CHAF1B, MOV10, CAB39L, FN1, DDX19B, BUB1, GPSM2, MSH5, ETV7, SUN1, GRAMD1C, LACTB2, LOC652276, EXOSC10, NUP210, ACOX3, UNC5CL, GNAO1, CGN, ZC3H18, CTSC, MFSD13A, and CCDC183; and determining if the patient has a low risk, an intermediate risk, or a high risk of recurrence of PTC based on the level of expression of the two or more genes of the gene signature.

Other aspects and features of the methods of the present disclosure will become apparent to those ordinarily skilled in the art upon review of the following description of specific embodiments.

### BRIEF DESCRIPTION OF THE DRAWINGS

These and other features of the present disclosure will become more apparent in the following detailed description in which reference is made to the appended drawings. The appended drawings illustrate one or more embodiments of the present disclosure by way of example only and are not to be construed as limiting the scope of the present disclosure.

FIG. 1 shows Kaplan-Meier curves for patients classified using the methods of the present disclosure, wherein FIG. 1A shows the Kaplan-Meier curve for patients in a first cohort; FIG. 1B shows the Kaplan-Meier curve for patients in the first cohort classified in one embodiment of the present disclosure; and FIG. 1C shows the Kaplan-Meier curve for patients in the first cohort classified in another embodiment of the present disclosure.

FIG. 2 shows the Kaplan-Meier curve for patients in a second cohort classified using an embodiment of the present disclosure.

FIG. 3 shows a flowchart illustrating an embodiment of the present disclosure.

FIG. 4 shows a schematic diagram of a system for implementing an embodiment of the present disclosure.

FIG. 5 shows a schematic diagram of a hardware structure of a computing device of the system shown in FIG. 4.

FIG. 6 shows a schematic diagram of a simplified software architecture of a computing device of the system shown in FIG. 4.

FIG. 7 shows Kaplan-Meier curves for patients classified using the American Thyroid Association (ATA) Disease Recurrence Risk Stratification system, wherein FIG. 7A shows the Kaplan-Meier curve for patients in the first cohort of FIG. 1; and FIG. 7B shows the Kaplan-Meier curve for patients in the second cohort of FIG. 2.

FIG. 8 shows time-dependent area under the receiver operating characteristic curve (AUROC) graphs comparing an embodiment of the present disclosure with the American Thyroid Association (ATA) Disease Recurrence Risk Stratification system at a time of four years, wherein FIG. 8A shows the time-dependent AUROC for an embodiment of the present disclosure; and FIG. 8B shows the time-dependent AUROC for the ATA system.

FIG. 9 shows a graph of the percent recurrence for patients classified as having a low risk, an intermediate risk, or a high risk of recurrence by the American Thyroid Association (ATA) Disease Recurrence Risk Stratification system and an embodiment of present disclosure.

### DETAILED DESCRIPTION

The embodiments of the present disclosure generally relate to methods of determining the risk of recurrence of papillary thyroid cancer (PTC) in a patient as well as methods of treating such patients. The embodiments of the present disclosure also relate to systems for performing the methods described herein.

The methods of the present disclosure were developed as a result of extensive genomic research. In more detail, The Cancer Genome Atlas (TCGA) Network published the complete genomic landscape of PTC, which included a description of the molecular features of PTC as well as molecular subgroups identified using unsupervised clustering methods. Two meta-clusters were identified: one containing BRAF^{V600E}-driven tumors, and one containing tumors having Ras mutations. At the messenger ribonucleic acid (mRNA) level, the microRNA (miRNA) level, DNA methylation level and protein expression levels, the number of subgroups varied but were predominantly associated with one of the two meta-clusters. However, while TCGA provided insight into the molecular diversity and classification of PTC, the molecular subgroups were not related to potential clinical outcomes (i.e. prognosticating). Thus, there remains a need to identify genes that are related, either alone or in combination with others, to potential clinical outcomes for PTC patients.

In order to develop the methods of the present disclosure, extensive research was performed into the RNA-sequence expression dataset provided by TCGA, which contains batch-corrected expression levels of more than 22,000 genes from 502 PTC patient samples. From this expansive dataset, a gene signature was identified that comprises the potentially prognostically significant genes outlined in Table 1.

TABLE 1
Prognostically significant genes, the locations of
protein production function, and the types thereof

| | | Entrez | | Location of | |
|---|---|---|---|---|---|
| Gene | | Gene | Ensembl Stable | Protein | |
| Symbol | Gene Name | ID | ID | Product | Type(s) |
| ABCC6P1 | ATP binding | 653190 | ENSG00000256340 | Other | Other |
| | cassette | | | | |
| | subfamily C | | | | |
| | member 6 | | | | |
| | pseudogene 1 | | | | |
| ABCC8 | ATP binding | 6833 | ENSG00000006071 | Plasma | Transporter |
| | cassette | | | Membrane | |
| | subfamily C | | | | |
| | member 8 | | | | |
| ACOX3 | Acyl-coa oxidase | 8310 | ENSG00000087008 | Cytoplasm | Enzyme |
| | 3, pristanoyl | | | | |
| AGFG2 | Arfgap with FG | 3268 | ENSG00000106351 | Other | Other |
| | repeats 2 | | | | |
| ASPHD1 | Aspartate beta- | 253982 | ENSG00000174939 | Other | Other |
| | hydroxylase | | | | |
| | domain | | | | |
| | containing 1 | | | | |
| ATG14 | Autophagy | 22863 | ENSG00000126775 | Cytoplasm | Other |
| | related 14 | | | | |
| ATP1B1 | Atpase Na+/K+ | 481 | ENSG00000143153 | Plasma | Transporter |
| | transporting | | | Membrane | |
| | subunit beta 1 | | | | |
| BNIP3 | BCL2 interacting | 664 | ENSG00000176171 | Cytoplasm | Other |
| | protein 3 | | | | |
| BUB1 | BUB1 mitotic | 699 | ENSG00000169679 | Nucleus | Kinase |
| | checkpoint | | | | |
| | serine/threonine | | | | |
| | kinase | | | | |
| C12orf76 | Chromosome 12 | 400073 | ENSG00000174456 | Other | Other |
| | open reading | | | | |
| | frame 76 | | | | |
| C2orf88 | Chromosome 2 | 84281 | ENSG00000187699 | Other | Other |
| | open reading | | | | |
| | frame 88 | | | | |
| CAB39L | Calcium binding | 81617 | ENSG00000102547 | Cytoplasm | Kinase |
| | protein 39 like | | | | |
| CCDC183 | Coiled-coil | 84960 | ENSG00000213213 | Other | Other |
| | domain | | | | |
| | containing 183 | | | | |
| CCNA2 | Cyclin A2 | 890 | ENSG00000145386 | Nucleus | Other |
| CDCA8 | Cell division cycle | 55143 | ENSG00000134690 | Nucleus | Other |
| | associated 8 | | | | |
| CENPL | Centromere | 91687 | ENSG00000120334 | Cytoplasm | Other |
| | protein L | | | | |
| CGN | Cingulin | 57530 | ENSG00000143375 | Plasma | Other |
| | | | | Membrane | |
| CHAF1B | Chromatin | 8208 | ENSG00000159259 | Nucleus | Other |
| | assembly factor 1 | | | | |
| | subunit B | | | | |
| CLDN12 | Claudin 12 | 9069 | ENSG00000157224 | Plasma | Other |
| | | | | Membrane | |
| COPS2 | COP9 | 9318 | ENSG00000166200 | Cytoplasm | Other |
| | signalosome | | | | |
| | subunit 2 | | | | |
| CTSC | Cathepsin C | 1075 | ENSG00000109861 | Cytoplasm | Peptidase |
| DDX19B | DEAD-box | 11269 | ENSG00000157349 | Nucleus | Enzyme |
| | helicase 19B | | | | |
| DISP1 | Dispatched RND | 84976 | ENSG00000154309 | Plasma | Transporter |
| | transporter family | | | Membrane | |
| | member 1 | | | | |
| DNAH11 | Dynein axonemal | 8701 | ENSG00000105877 | Cytoplasm | Enzyme |
| | heavy chain 11 | | | | |
| EEF1A2 | Eukaryotic | 1917 | ENSG00000101210 | Cytoplasm | Translation |
| | translation | | | | regulator |
| | elongation factor | | | | |
| | 1 alpha 2 | | | | |
| EIF2A | Eukaryotic | 83939 | ENSG00000144895 | Cytoplasm | Translation |
| | translation | | | | regulator |
| | initiation factor | | | | |
| | 2A | | | | |
| ERCC5 | ERCC excision | 2073 | ENSG00000134899 | Nucleus | Enzyme |
| | repair 5, | | | | |
| | endonuclease | | | | |
| ETV7 | ETS variant | 51513 | ENSG00000010030 | Nucleus | Transcription |
| | transcription | | | | regulator |
| | factor 7 | | | | |
| EXOSC10 | Exosome | 5394 | ENSG00000171824 | Nucleus | Kinase |
| | component 10 | | | | |
| EZH2 | Enhancer of | 2146 | ENSG00000106462 | Nucleus | Transcription |
| | zeste 2 polycomb | | | | regulator |
| | repressive | | | | |
| | complex 2 | | | | |
| | subunit | | | | |
| FAM86C1P | Family with | 55199 | ENSG00000158483 | Other | Other |
| | sequence | | | | |
| | similarity 86 | | | | |
| | member C1, | | | | |
| | pseudogene | | | | |
| FBXO4 | F-box protein 4 | 26272 | ENSG00000151876 | Nucleus | Enzyme |
| FN1 | Fibronectin 1 | 2335 | ENSG00000115414 | Extracellular | Enzyme |
| | | | | Space | |
| GATAD1 | GATA zinc finger | 57798 | ENSG00000157259 | Nucleus | Transcription |
| | domain | | | | regulator |
| | containing 1 | | | | |
| GCFC2 | GC-rich | 6936 | ENSG00000005436 | Nucleus | Transcription |
| | sequence DNA- | | | | regulator |
| | binding factor 2 | | | | |
| GNAO1 | G protein subunit | 2775 | ENSG00000087258 | Plasma | Enzyme |
| | alpha o1 | | | Membrane | |
| GNG4 | G protein subunit | 2786 | ENSG00000282972 | Plasma | Enzyme |
| | gamma 4 | | | Membrane | |
| GPSM2 | G protein | 29899 | ENSG00000121957 | Nucleus | Other |
| | signaling | | | | |
| | modulator 2 | | | | |
| GRAMD1C | GRAM domain | 54762 | ENSG00000178075 | Other | Other |
| | containing 1C | | | | |
| GTPBP2 | GTP binding | 54676 | ENSG00000172432 | Extracellular | Enzyme |
| | protein 2 | | | Space | |
| GTPBP8 | GTP binding | 29083 | ENSG00000163607 | Cytoplasm | Other |
| | protein 8 | | | | |
| | (putative) | | | | |
| HIST2H2BF | H2B clustered | 440689 | ENSG00000203814 | Nucleus | Other |
| | histone 18 | | | | |
| HIST4H4 | H4 histone 16 | 121504 | ENSG00000197837 | Nucleus | Other |
| HJURP | Holliday junction | 55355 | ENSG00000123485 | Nucleus | Other |
| | recognition | | | | |
| | protein | | | | |
| KHNYN | KH and NYN | 23351 | ENSG00000100441 | Other | Other |
| | domain | | | | |
| | containing | | | | |
| KIF4A | Kinesin family | 24137 | ENSG00000090889 | Nucleus | Other |
| | member 4A | | | | |
| LACTB2 | Lactamase beta | 51110 | ENSG00000147592 | Cytoplasm | Enzyme |
| | 2 | | | | |
| LANCL2 | Lanc like 2 | 55915 | ENSG00000132434 | Plasma | Other |
| | | | | Membrane | |
| LOC652276 | Potassium | 652276 | ENSG00000215154 | Other | Other |
| | channel | | | | |
| | tetramerization | | | | |
| | domain | | | | |
| | containing 5 | | | | |
| | pseudogene | | | | |
| LOC728613 | Programmed cell | 728613 | N/A | Other | Other |
| | death 6 | | | | |
| | pseudogene | | | | |
| LTK | Leukocyte | 4058 | ENSG00000062524 | Plasma | Kinase |
| | receptor tyrosine | | | Membrane | |
| | kinase | | | | |
| MFSD13A | Major facilitator | 79847 | ENSG00000138111 | Other | Other |
| | superfamily | | | | |
| | domain | | | | |
| | containing 13A | | | | |
| MOV10 | Mov10 RISC | 4343 | ENSG00000155363 | Nucleus | Enzyme |
| | complex RNA | | | | |
| | helicase | | | | |
| MSH5 | Muts homolog 5 | 4439 | ENSG00000233345 | Nucleus | Enzyme |
| MTMR14 | Myotubularin | 64419 | ENSG00000163719 | Cytoplasm | Phosphatase |
| | related protein 14 | | | | |
| MUC21 | Mucin 21, cell | 394263 | ENSG00000231350 | Cytoplasm | Other |
| | surface | | | | |
| | associated | | | | |
| MYO3A | Myosin IIIA | 53904 | ENSG00000095777 | Cytoplasm | Kinase |
| NEK2 | NIMA related | 4751 | ENSG00000117650 | Cytoplasm | Kinase |
| | kinase 2 | | | | |
| NFATC2IP | Nuclear factor of | 84901 | ENSG00000176953 | Nucleus | Other |
| | activated T cells | | | | |
| | 2 interacting | | | | |
| | protein | | | | |
| NUDT15 | Nudix hydrolase | 55270 | ENSG00000136159 | Cytoplasm | Phosphatase |
| | 15 | | | | |
| NUP210 | Nucleoporin 210 | 23225 | ENSG00000132182 | Nucleus | Transporter |
| PGBD5 | Piggybac | 79605 | ENSG00000177614 | Nucleus | Enzyme |
| | transposable | | | | |
| | element derived | | | | |
| | 5 | | | | |
| REP15 | RAB15 effector | 387849 | ENSG00000174236 | Cytoplasm | Other |
| | protein | | | | |
| REXO5 | RNA | 81691 | ENSG00000005189 | Nucleus | Enzyme |
| | exonuclease 5 | | | | |
| RHBDF1 | Rhomboid 5 | 64285 | ENSG00000007384 | Cytoplasm | Other |
| | homolog 1 | | | | |
| RPRM | Reprimo, TP53 | 56475 | ENSG00000177519 | Cytoplasm | Other |
| | dependent G2 | | | | |
| | arrest mediator | | | | |
| | homolog | | | | |
| RRAGA | Ras related GTP | 10670 | ENSG00000155876 | Cytoplasm | Enzyme |
| | binding A | | | | |
| SEPSECS | Sep (O- | 51091 | ENSG00000109618 | Cytoplasm | Enzyme |
| | phosphoserine) | | | | |
| | trna:Sec | | | | |
| | (selenocysteine) | | | | |
| | trna synthase | | | | |
| SKA3 | Spindle and | 221150 | ENSG00000165480 | Nucleus | Other |
| | kinetochore | | | | |
| | associated | | | | |
| | complex subunit | | | | |
| | 3 | | | | |
| SLC43A1 | Solute carrier | 8501 | ENSG00000149150 | Plasma | Transporter |
| | family 43 | | | Membrane | |
| | member 1 | | | | |
| SNX29P2 | Sorting nexin 29 | 440352 | ENSG00000271699 | Other | Other |
| | pseudogene 2 | | | | |
| SUN1 | Sad1 and UNC84 | 23353 | ENSG00000164828 | Nucleus | Other |
| | domain | | | | |
| | containing 1 | | | | |
| TAFA2 | TAFA chemokine | 338811 | ENSG00000198673 | Cytoplasm | Other |
| | like family | | | | |
| | member 2 | | | | |
| TICRR | TOPBP1 | 90381 | ENSG00000140534 | Nucleus | Other |
| | interacting | | | | |
| | checkpoint and | | | | |
| | replication | | | | |
| | regulator | | | | |
| TTK | TTK protein | 7272 | ENSG00000112742 | Nucleus | Kinase |
| | kinase | | | | |
| TXNL4B | Thioredoxin like | 54957 | ENSG00000140830 | Nucleus | Enzyme |
| | 4B | | | | |
| UNC5CL | Unc-5 family C- | 222643 | ENSG00000124602 | Cytoplasm | Peptidase |
| | terminal like | | | | |
| WDR1 | WD repeat | 9948 | ENSG00000071127 | Extracellular | Other |
| | domain 1 | | | Space | |
| WWC3 | WWC family | 55841 | ENSG00000047644 | Cytoplasm | Other |
| | member 3 | | | | |
| ZC3H18 | Zinc finger | 124245 | ENSG00000158545 | Nucleus | Other |
| | CCCH-type | | | | |
| | containing 18 | | | | |
| ZNF215 | Zinc finger | 7762 | ENSG00000149054 | Nucleus | Transcription |
| | protein 215 | | | | regulator |
| ZNF620 | Zinc finger | 253639 | ENSG00000177842 | Nucleus | Transcription |
| | protein 620 | | | | regulator |

This specific gene signature, to the knowledge of the inventors, has not previously been used for the prognosis of PTC.

In more detail, the gene signature of the present disclosure was acquired using the following procedure. As indicated above, TCGA contains batch-corrected expression levels of more than 22,000 genes and accompanying clinical outcomes including progression-free survival (i.e. recurrence information) from 502 PTC patient samples. The 502 PTC patient samples were divided into a first cohort containing 335 samples and a second cohort containing 167 samples. The first cohort was used to determine the gene signature of the present disclosure and to train a statistical model to classify patients as having a low risk, an intermediate risk, or a high risk of PTC recurrence. The second cohort was used for independent validation of the gene signature and the statistical model. In total, the associations of genes were tested in more than 12,824,240 combinations of genes and cohorts in order to identify the prognostically significant genes of the gene signature of the present disclosure.

The first cohort was examined to identify a first set of prognostically significant genes: ATG14, MYO3A, ERCC5, SLC43A1, ABCC8, LTK, COPS2, CCNA2, BNIP3, FAM86C1P, GNG4, GCFC2, EEF1A2, TXNL4B, SEPSECS, ZNF215, KIF4A, EZH2, CDCA8, DISP1, SNX29P2, ATP1B1, ZNF620, HIST4H4, CENPL, GATAD1, C2orf88, WWC3, SKA3, HJURP, LOC728613, GTPBP8, RPRM, FBXO4, TICRR, AGFG2, TTK, TAFA2, MTMR14, WDR1, NEK2, RRAGA, EIF2A, and REP15. Then, using non-censored members of the first cohort that experienced a recurrence of PTC or that were disease-free after at least 36 months of follow-up (N=222), a second set of prognostically significant genes were identified: NUDT15, LANCL2, NFATC2IP, GTPBP2, ZNF215, KHNYN, CLDN12, DNAH11, EZH2, ASPHD1, REXO5, HIST2H2BF, C12orf76, MUC21, PGBD5, ABCC6P1, RHBDF1, CHAF1B, MOV10, CAB39L, FN1, DDX19B, BUB1, GPSM2, MSH5, ETV7, SUN1, MTMR14, GRAMD1C, LACTB2, LOC652276, EXOSC10, NUP210, ACOX3, UNC5CL, GNAO1, CGN, ZC3H18, CTSC, MFSD13A, and CCDC183. Of the second gene set, only three genes overlapped with the first gene set, namely EZH2, MTMR14, and ZNF215.

The two gene sets were combined to provide the gene signature of the present disclosure identified in Table 1. Notably, as shown in Table 1, there is no clearly identifiable pattern in the location of protein product, functionality or type of genes of the gene signature of the present disclosure. That is, the locations and types of the genes of the identified gene signature are generally disparate.

Using the gene signature of the present disclosure, it became possible to classify the patients of the first cohort into three distinct prognostic groups based on their risk of recurrence of PTC, namely a low-risk group, an intermediate-risk group, and a high-risk group. In more detail, a statistical model for classifying the patients was trained using expression data of the genes in the gene signature of the present disclosure from the patients of the first cohort. Training the statistical model generally involved analyzing the performance of various models, which may be quantified by the true positive rates, false negative rates, precision, mean absolute error, root mean squared error, root relative squared error, and the confusion matrices of the various models, detailing correctly and incorrectly classified patients, and adjusting the models based on the results of the analyses. The three prognostic groups identified were distinct in that they had statistically different (log rank p<0.0001) probabilities of progression-free survival (i.e. the length of time during and after the treatment of the disease that the patient lives with the disease without it getting worse).

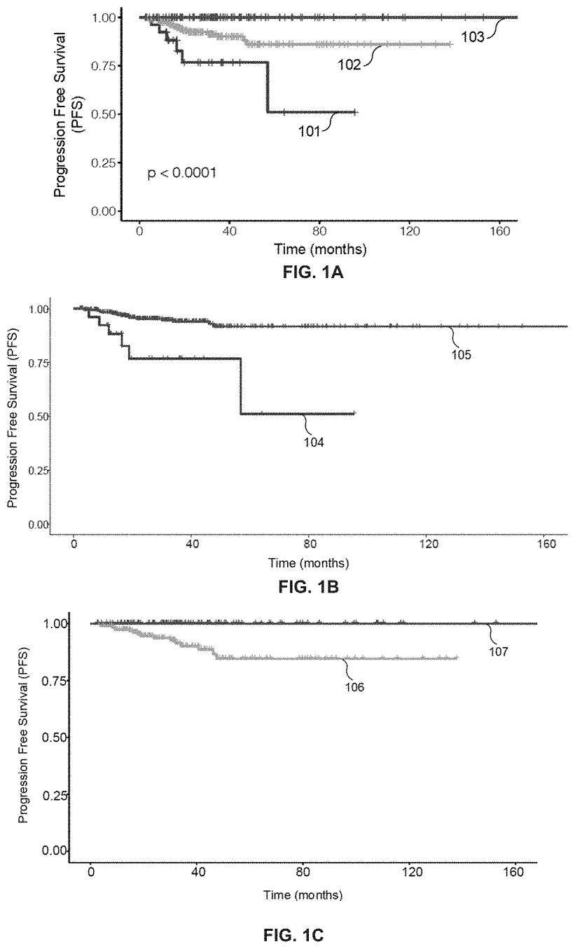

In more detail, by determining the level of expression of two or more genes of the gene signature of the present disclosure, the patients of the first cohort were classified, using a statistical model as described above, into a group having a high risk of PTC recurrence, an intermediate-risk of PTC recurrence, and a low-risk of PTC recurrence, as shown FIG. 1A, wherein the line 101 represents the high-risk group, the line 102 represents the intermediate-risk group, and the line 103 represents the low-risk-group.

As well, it was found that patients may be classified into risk strata using the gene signature of the present disclosure in a series of steps. For example, again using the first cohort, the level of expression of two or more genes of the first set of prognostically significant genes was determined to identify, using the statistical model, a group having a high risk of PTC recurrence (line 104) and a group having a non-high risk of PTC recurrence (line 105), as shown in FIG. 1B. Then, by determining the level of expression or two or more genes of the second set of prognostically significant genes, within the group having a non-high risk of recurrence there was identified, using the statistical model, a group having a low-risk of PTC recurrence (line 107), with the remaining patients forming a group having an intermediate-risk of PTC recurrence (line 106), as shown in FIG. 1C.

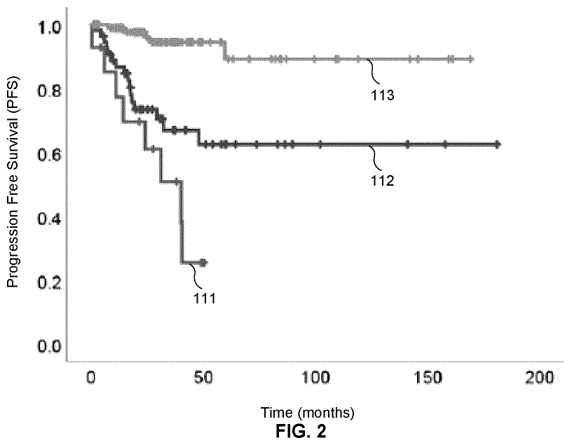

Using the second cohort, the gene signature and statistical model were independently validated. Like the first cohort, the level of expression of two or more of the genes of the gene signature of the present disclosure was measured, and the patients were classified as having a low risk, an intermediate risk, or a high risk of PTC recurrence using the statistical model trained using the patients of the first cohort. Again, each of the three prognostic groups were statistically distinct (log rank p<0.0001) in relation to progression-free survival (PFS), as illustrated in FIG. 2, wherein the line 111 represents the high-risk group, the line 112 represents the intermediate-risk group, and the line 113 represents the low-risk-group.

The inventors also investigated the clinical and molecular differences between the three prognostic groups. Using a combination of the first and second cohorts, it was found that 32.4% of the patients belonged to the low-risk group, 59.3% of the patients belonged to the intermediate-risk group, and 8.3% of the patients belonged to the high-risk group. Notably, the inventors found no significant relationship between risk and sex, race, ethnicity, stage, tumor size, lymph node status or histological variant. However, it was found that both age, distant metastases, extent, and size (AMES) scores and distant metastasis, completeness of resection, local invasion, and tumor size (MACIS) scores increased from the low-risk group to the high-risk group.

Further, the inventors discovered trends within each of the risk groups. For example, tumors of the high-risk group were generally characterized by de-differentiation, enrichment of the EZH2-Noxa transcript antisense RNA pathway (EZH2-HOTAIR pathway), and an inflamed but immunosuppressed microenvironment. The tumors of the intermediate-risk group could actually be separated into two distinct subtypes having the same risk of PTC recurrence: a first intermediate-risk subtype having a high prevalence of BRAF^{V600E} mutations (“BRAF_{HIGH}” subtype) and a second intermediate-risk subtype enriched with RAS mutations and having few BRAF^{V600E} mutations (“BRAF_{LOW}” subtype). Such discoveries may be useful in selecting and administering treatments to patients with PTC.

In more detail, Ingenuity Pathway Analysis (IPA) showed that the tumors in the high-risk group of patients had a significant enrichment of genes involved in HMGB1 signaling, Stat3 signaling, IL-23 signaling, IL-17 signaling, and NF-κB signaling. Without being bound to any particular theory, HMGB1 upregulation and successive elaboration of IL-23, IL-17 and IL-6 followed by Stat3 activation may promote tumor growth. It also appears that Stat3, in tumor and myeloid cells, may induce IL-23 production by tumor-associated macrophages. Regulatory T cells expressing IL-23R may then be activated to create the immunosuppressive tumor microenvironment described above.

Further, deconvolution of immune components revealed that the tumors of the high-risk group had a higher lymphocyte infiltration score. These tumors had higher numbers of resting CD4+ memory cells, naïve B cells, follicular helper T cells, and regulatory T cells. M1 macrophage infiltration was greater while M2 macrophage content was less.

The IPA also showed the positive enrichment of the HOXA transcript antisense RNA (HOTAIR) pathway, which is a long non-coding RNA (lncRNA) that interacts with Polycomb Repressive Complex 2 (PRC2), a histone methyltransferase that affects epigenetic silencing supporting diverse proneoplastic processes including epithelial-to-mesenchymal transition (EMT). The HOTAIR interaction with PRC2 drives EZH2-mediated gene repression. Elevated EZH2 expression may be characteristic of tumors having a high-risk of recurrence. As well, HOTAIR myeloid-specific 1 (HOTAIRM1), which similarly interacts with EZH2 and which may also encourage an immunosuppressive microenvironment, was also upregulated.

In comparison with the tumors of patients in the low-risk group, the tumors of the high-risk group generally included a significantly greater number of hypermethylated genes. Of 61 differentially methylated genes, LINC00310, HOXA10, VWA3A, SMOC2, APLP2, SLC38A4, SLC10A6, PLCH1, CFAP73, ADGRL2, LINC01091, and CPQ had corresponding significant downregulation at the transcriptional level. Without being bound to any particular theory, LINC00310 may be associated with cancer recurrence when expression levels are decreased and expression levels of MAPK10 may be downregulated in anaplastic thyroid cancers.

There were 4 hypomethylated genes associated with significant upregulated gene expression, including HLA-DMA, which may correlate with PD-L1 expression in ovarian cancer. There were also 100 differentially expressed micro RNAs (miRNAs), of which 96 miRNAs had higher expression levels in the high-risk group and had 273 downregulated mRNA targets, and 4 miRNAs, namely hsa-mir-450b, hsa-mir-346, hsa-mir-483, and hsa-mir-1251, were less abundant in the high-risk group and had 47 upregulated mRNA targets. Many of the upregulated genes in the high-risk group that were associated with downregulated miRNAs had inflammatory and immune functions, such genes including, for example, CD4, IL10RA, CD247, IL21R, and TRAT1.

With respect to the intermediate-risk group, a first subgroup was highly enriched with BRAF^{V600E} mutations (BRAF_{HIGH}) and contained all of the tumors with a tall cell variant histology. A second subgroup was enriched with RAS mutations (BRAF_{LOW}). The BRAF_{HIGH} subgroup had a significantly lower thyroid differentiation index (TDI) than the BRAF_{low} subgroup. As will be appreciated by those skilled in the art, the TDI was determined by TGCA and reflects the expression levels of 16 thyroid metabolism and function genes, namely DIO1, DIO2, DUOX1, DUOX2, FOXE1, GLIS3, NKX2-1, PAX8, SLC26A4, SLC5AA5, SLC5A8, TG, THRA, THRB, TPO, and TSHR. In general, a lower TDI reflects a higher histological grade, which may imply a greater de-differentiation of cancer cells. Further, clinically, BRAF_{HIGH} tumors were higher in tumor, lymph node, and metastasis (TNM) stage according to the TNM Classification of Malignant Tumors, had a higher prevalence of extrathyroidal extension, more frequently had lymph node metastases, and generally had a higher ATA risk classification. The BRAF_{LOW} subgroup, which included most of the follicular variants, was significantly enriched with NRAS and HRAS mutations. Mutations in the thyroglobulin gene were also significantly more common in the BRAF_{LOW} subgroup. Further, EIF1AX mutations were exclusively found in the BRAF_{LOW} subgroup.

The biological features of the two intermediate-risk subgroups were also different. For example, the BRAF_{HIGH} subgroup demonstrated significant positive enrichment in proinflammatory genes, genes involved in angiogenesis and EMT, as well as genes associated with estrogen response. The BRAF_{HIGH} also demonstrated many of the features of the high-risk group, however to a lesser extent. As well, there was positive enrichment in genes associated with dendritic cell maturation, IL-17 signaling, and Th1 and Th2 activation. With respect to the BRAF_{LOW} subgroup, the HOTAIR regulatory pathway was not dysregulated and was instead characterized by metabolic features including alterations in lipid metabolism such as β-oxidation of fatty acids. Further, in general, the BRAF_{LOW} subgroup also had significantly more hypermethylated genes than all other groups.

Relative to the low-risk group, both intermediate risk subgroups had significantly more differentially expressed miRNAs. In more detail, the inventors found that there were 1013 unique upregulated miRNA and downregulated mRNA target combinations, and 822 unique downregulated miRNA and upregulated mRNA target combinations. Without being bound to any particular theory, miRNA targets in the BRAF_{LOW} subgroup may suggest decreased inflammatory signaling. For example, IL31RA, IL1RAP, IL11, IL2RA, and IL7R were downregulated mRNA targets in the BRAF_{LOW} subgroup. In the BRAF_{HIGH} subgroup, the inventors found that there were 1500 unique upregulated miRNA and downregulated mRNA target combinations, and 609 unique downregulated miRNA and upregulated mRNA target combinations. As a result of differential expression of miRNAs, there was increased expression of CD28, HLA-A, HLA-DRB1, HLA-DRA, HLA-DRB5, HLA-DOA, CD3D, CD3G, IL10, IL21R, and CD40LG in the BRAF_{HIGH} subgroup, which may indicate that genes involved in inflammatory and immune processes were predominately targeted.

As indicated by the experimental results discussed below, the methods of the present disclosure may provide more accurate estimates of whether a patient has a low risk, an intermediate risk, or a high risk of PTC recurrence as compared to conventional methods—i.e. those used by the American Thyroid Association (ATA) Disease Recurrence Risk Stratification system.

The accurate prognostication of PTC affords several advantages. For example, accurate identification of low-risk or intermediate-risk PTC may result in a patient being treated with active surveillance or a hemithyroidectomy, rather than a total thyroidectomy as required in aggressive cases of PTC. This is advantageous for a number of reasons. Firstly, such patients avoid the need for life-long replacement of thyroid hormones, which is required after total thyroidectomies. Secondly, active surveillance and hemithyroidectomy each present a greatly reduced risk of the potentially serious complications associated with total thyroidectomies. Such complications include bilateral recurrent laryngeal nerve injury and permanent hypoparathyroidism.

Further, accurate identification low-risk and intermediate-risk PTC may also aid in the determination of whether adjuvant radioactive-iodine (RAI) is appropriate. RAI therapy not only requires significant resources and costs, but may also result in long term, morbid side-effects. Such side effects include salivary gland dysfunction, premature menopause, and testicular failure. As well, RAI therapy may also result in secondary malignancy—i.e. cancer caused by the radioactive treatment.

Additionally, accurately identifying low-risk and intermediate-risk PTC may also affect the degree of active surveillance that a patient receives. Active surveillance may involve regular examination in order to detect early signs of recurrence, which may continue for many years. As well, follow-up examinations typically involve annual physical examinations, serum measurements of thyroid-stimulating hormone and thyroglobulin, as well as periodic neck ultrasounds. As will be appreciated by the skilled person, the many aspects of active surveillance may burden both the patient and the healthcare organization administering the active surveillance. However, patients with, for example, low-risk PTC may require fewer follow-up examinations and, in some cases, may be discharged from active surveillance, thereby reducing the resource and financial burdens placed on the patient as well as the healthcare organization.

Furthermore, the methods of the present disclosure may afford the accurate determination of high-risk PTC in a patient. As a result, patients may be administered an appropriate treatment (i.e. one that is aggressive enough to fully treat the PTC), thereby avoiding a situation where they are undertreated.

In addition to accurately determining whether a patient has a low risk, an intermediate risk, or a high risk of PTC recurrence, the methods of the present disclosure may be used to determine the type of treatment most suitable for patients with an intermediate risk of PTC recurrence. As described herein, there are a number of treatments that may be selected and administered to patients with an intermediate risk of PTC recurrence. However, depending on the gene expression profile of a tumor of the patient, certain types of treatments may be more effective than others. For example, tumors of intermediate-risk patients that have a high prevalence of BRAF^{V600E} mutations may be resistant to RAI while having an increased sensitivity to EZH2 inhibitors and immune checkpoint inhibitors. In contrast, tumors of intermediate-risk patients that have few BRAF^{V600E} mutations and are enriched with RAS mutations may be more susceptible to RAI.

In view of the above, some embodiments of the present disclosure relate to a method of determining the risk of recurrence of papillary thyroid cancer (PTC) in a patient, the method comprising: isolating RNA from a biological sample of the patient; determining from the RNA the level of expression of two or more genes or gene products of a gene signature comprising: ATG14, MYO3A, ERCC5, SLC43A1, ABCC8, LTK, COPS2, CCNA2, BNIP3, FAM86C1P, GNG4, GCFC2, EEF1A2, TXNL4B, SEPSECS, ZNF215, KIF4A, EZH2, CDCA8, DISP1, SNX29P2, ATP1B1, ZNF620, HIST4H4, CENPL, GATAD1, C2orf88, WWC3, SKA3, HJURP, LOC728613, GTPBP8, RPRM, FBXO4, TICRR, AGFG2, TTK, TAFA2, MTMR14, WDR1, NEK2, RRAGA, EIF2A, REP15, NUDT15, LANCL2, NFATC2IP, GTPBP2, KHNYN, CLDN12, DNAH11, ASPHD1, REXO5, HIST2H2BF, C12orf76, MUC21, PGBD5, ABCC6P1, RHBDF1, CHAF1B, MOV10, CAB39L, FN1, DDX19B, BUB1, GPSM2, MSH5, ETV7, SUN1, GRAMD1C, LACTB2, LOC652276, EXOSC10, NUP210, ACOX3, UNC5CL, GNAO1, CGN, ZC3H18, CTSC, MFSD13A, and CCDC183; and, determining whether the patient has a low risk, intermediate risk, or high risk of PTC recurrence based on the level of expression of the two or more genes or gene products of the gene signature.

The biological sample may be obtained by macrodissection or microdissection of a tumor. In general, microdissections encompass dissections that involve the use of a microscope to collect a sample, while macrodissections encompass dissections that do not involve the use of a microscope. Suitable dissection techniques include, without limitation, laser capture microdissection, pressure catapulting, or combinations thereof. Laser capture microdissection involves the use of a laser through a microscope to cause selected cells to adhere to a film. Pressure catapulting involves catapulting cells into a collection vessel without physically contacting the cells.

In some embodiments of the present disclosure, the tumor may be a formalin-fixed paraffin embedded (FFPE) sample or a frozen biopsy sample. In some embodiments, the tumor may be a sample obtained by a fine-needle aspiration, a core biopsy, or from a surgical specimen. In more detail, fine-needle aspiration includes inserting a thin (e.g. a diameter of 0.52 mm to 64 mm) hollow needle into the mass of the tumor and withdrawing cells therefrom via aspiration. A core biopsy is similar to that of the fine needle aspiration but uses a larger needle (e.g. a diameter of 1.02 mm to 2.3 mm). In regards to the surgical specimen, the specimen may have been obtained, for example, by a previously performed thyroid resection.

The isolating of RNA from the tumor may be done in vitro using various techniques such as cesium chloride density gradient centrifugation. Cesium chloride density gradient involves centrifuging a solution containing cesium chloride and a sample comprising DNA and/or RNA productions. During centrifuging, the cesium ions, due to their weight, will move from the center towards the outer end of vessel while, at same time, diffusing back towards the top of the vessel, thereby forming a shallow density gradient. DNA and/or RNA products present in the solution will migrate to the point at which they have the same density as the gradient (i.e. neutral buoyancy or their isopycnic point), thereby separating.

The isolation of the RNA from the tumor may also be done in vitro using techniques such as acid guanidinium thiocyanate-phenol-chloroform extraction (AGPC). AGPC involves centrifugation of a mixture of an aqueous sample and a solution containing water-saturated phenol and chloroform, which produces an upper aqueous phase and a lower organic phase that comprises mainly phenol. Guanidinium thiocyanate is added to the organic phase to facilitate the denaturation of proteins (e.g. those that degrade RNA). The nucleic acids partition into the aqueous phase, while protein partitions into the organic phase. The pH of the mixture determines which nucleic acids get purified. For example, under acidic conditions (e.g. a pH of 4 to 6), DNA partitions into the organic phase while RNA remains in the aqueous phase. In a last step, the nucleic acids are recovered from the aqueous phase by precipitation with a solvent such as 2-propanol.

The isolation of the RNA from the tumor may also be done in vitro using techniques such as spin-column based nucleic acid purification. Spin-column based nucleic acid purification may employ a silica-gel membrane for the selective absorption of nucleic acids. In more detail, the cells of a sample are first lysed to remove the nucleic acid therefrom. A buffer solution is then added to the sample with a solvent such as ethanol or isopropanol to form a binding solution. The binding solution is transferred to a spin column and subsequently centrifuged, which causes the binding solution to pass through a silica-gel membrane inside the spin column to thereby bind nucleic acids contained in the binding solution to the membrane. The centrifuged binding solution is then removed so that the silica-gel membrane may be washed and the nucleic acids eluted. To wash the silica gel membrane, the spin column is centrifuged with a wash buffer to remove any impurities bound the silica gel. To elute, the wash buffer is removed and the spin column is centrifuged with an elution buffer (e.g. water) to remove the nucleic acid from the membrane for collection at the bottom of the spin column.

Once the RNA is isolated, the level of expression of the two or more genes of the gene signature of the present disclosure may be determined. The level of expression of each gene of the gene signature of the present disclosure may be determined by, for example, reverse-transcription polymerase chain reaction (RT-PCR). RT-PCR generally involves reverse transcription of the RNA template into complementary DNA (cDNA) and subsequent amplification via a PCR reaction. For the reverse transcription, enzymes such as avian myeloblastosis virus reverse transcriptase (AMV-RT) and Moloney murine leukemia virus reverse transcriptase (MMLV-RT) may be used. The reverse transcription may be primed using random hexamers, oligo-dT primers, and the like. In regards to the PCR reaction, a variety of thermostable DNA-dependent DNA polymerases may be used. One example of a suitable DNA polymerase includes Taq DNA polymerase, which has a 5′-3′ nuclease activity but lacks 3′-5′ proofreading endonuclease activity.

Other platforms for determining the level of gene expression of the two or more genes of the gene signature may also be used. For example, such platforms include cDNA microarrays, RNAseq, and nCounter™ DX analysis systems provided by Nanostring.

As described above, the methods of the present disclosure may involve determining the levels of expression of two or more gene products of the gene signature. In some embodiments, the gene products may be proteins formed from translation of a transcribed gene of the gene signature. The levels of expression of the proteins may be determined using any suitable technique, including, for example, an ultraviolet absorption method, a Biuret method (e.g. a bicinchoninic acid assay or a Lowry assay), a colorimetric dye-based method (e.g. a Bradford assay), a fluorescent dye method, a proteomic method (e.g. mass spectrometry-based methods), or any combination thereof.

In some embodiments, the determining of the level of expression of the two or more genes of the gene signature comprises determining the level of expression of three or more genes of the gene signature. In one embodiment the determining of the level of expression of the two or more genes of the gene signature comprises determining the level of expression of four or five or six or seven or eight or nine or ten or more genes of the gene signature. In another embodiment, the determining of the level of expression of the two or more genes of the gene signature comprises determining the level of expression of 20 to 60 genes of the gene signature. In a further embodiment, the determining of the level of expression of the two or more genes of the gene signature comprises determining the level of expression of 20 to 50, 30 to 60, 40 to 60, or 40 to 50 genes of the gene signature. In a particular embodiment, the determining of the level of expression of the two or more genes or gene products of the gene signature comprises determining the level of expression of at least the genes ATG14, MYO3A, ERCC5, SLC43A1, ABCC8, LTK, COPS2, CCNA2, BNIP3, FAM86C1P, GNG4, GCFC2, EEF1A2, TXNL4B, SEPSECS, ZNF215, KIF4A, EZH2, CDCA8, DISP1, SNX29P2, ATP1B1, ZNF620, NUDT15, LANCL2, NFATC2IP, GTPBP2, ZNF215, KHNYN, CLDN12, DNAH11, EZH2, ASPHD1, REXO5, HIST2H2BF, C12orf76, MUC21, PGBD5, ABCC6P1, RHBDF1, CHAF1B, MOV10, CAB39L, FN1, DDX19B, and BUB1.

In some embodiments, the determining of the level of expression of the two or more genes of the gene signature comprises a first step of determining the level of expression of a first gene set, and a second step of determining the level of expression of a second gene set. In some embodiments, first step comprises the determining of the level of expression of the two or more genes of the gene signature comprises determining the level of expression of three or four or five or six or seven or eight or nine or ten or more genes of the gene signature. In one embodiment, the first step comprises determining the level of expression of between about 20 genes to about 60 genes, between about 20 genes to about 50 genes, between about 30 genes to about 60 genes, between about 30 genes to about 50 genes, or between about 40 genes to about 50 genes of the gene signature.

In some embodiments, the first gene set comprises: ATG14, MYO3A, ERCC5, SLC43A1, ABCC8, LTK, COPS2, CCNA2, BNIP3, FAM86C1P, GNG4, GCFC2, EEF1A2, TXNL4B, SEPSECS, ZNF215, KIF4A, EZH2, CDCA8, DISP1, SNX29P2, ATP1B1, ZNF620, HIST4H4, CENPL, GATAD1, C2orf88, WWC3, SKA3, HJURP, LOC728613, GTPBP8, RPRM, FBXO4, TICRR, AGFG2, TTK, TAFA2, MTMR14, WDR1, NEK2, RRAGA, EIF2A, and REP15. In such embodiments, the first step may comprise determining the level of two or more genes of the first gene set. In a further embodiment, the first step comprises determining the level of expression of at least the following genes of the first gene set: ATG14, MYO3A, ERCC5, SLC43A1, ABCC8, LTK, COPS2, CCNA2, BNIP3, FAM86C1P, GNG4, GCFC2, EEF1A2, TXNL4B, SEPSECS, ZNF215, KIF4A, EZH2, CDCA8, DISP1, SNX29P2, ATP1B1, and ZNF620.

In some embodiments, the second gene set comprises the gene signature of the present disclosure. Thus, in one embodiment, the second step comprises determining the level of expression of three or four or five or six or seven or eight or nine or ten or more genes or gene products of the gene signature. In some embodiments, the second step comprises determining the level of expression of between about 20 genes to about 60 genes, between about 20 genes to about 50 genes, between about 30 genes to about 60 genes, between about 30 genes to about 50 genes, or between about 30 genes to about 50 genes of the gene signature. In a further embodiment, the second step comprises determining the level of expression of at least the following genes or gene products of the gene signature: NUDT15, LANCL2, NFATC2IP, GTPBP2, ZNF215, KHNYN, CLDN12, DNAH11, EZH2, ASPHD1, REXO5, HIST2H2BF, C12orf76, MUC21, PGBD5, ABCC6P1, RHBDF1, CHAF1B, MOV10, CAB39L, FN1, DDX19B, and BUB1.

In some embodiments, the step of determining if the patient has a low risk, an intermediate risk, or a high risk of recurrence of PTC based on the level of expression of the two or more genes of the gene signature comprises using the determined levels of expression and a statistical model for predicting the risk of recurrence of PTC in the patient. As described above, the statistical model may be trained using the expression levels of the genes of the gene signature of the present disclosure from a plurality of patients in combination with corresponding recurrence data of the plurality of patients (e.g. the first cohort of the TGCA patient samples described above). A trained statistical model may be referred to broadly herein as a “predictor algorithm” or “classifier algorithm”. In some embodiments, the predictor or classifier algorithm may comprise a statistical model such as a regression-based model (e.g. a logistic regression model), a machine learning algorithm (e.g. decision-tree based algorithms such as random forests, Bayes' theorem-based algorithms such as Naïve Bayes classifiers, k-nearest neighbors-based algorithms such as radial basis function networks, support vector machines, and ensemble learning algorithms), or an artificial intelligence (e.g. artificial neural networks). In some embodiments, the predictor or classifier algorithm may compare the level of expression of the two or more genes or gene products of the gene signature to the levels of expression of the same genes or gene products of a patient previously determined to have a low risk of PTC recurrence.

Thus, in some embodiments, using the trained statistical model to determine the risk of recurrence of PTC in a patient may comprise providing the expression levels of two or more genes of the gene signature of the present disclosure into the statistical model to thereby determine the patient's risk of PTC recurrence. Further, the step of determining if the patient has a low risk, intermediate risk, or high risk of recurrence PTC based on the level of expression of the two or more genes or gene products of the gene signature may comprise dichotomizing (i.e. separating into two groups) using the expression levels of the first gene set. In such embodiments, the methods of the present disclosure may comprise determining if the patient has a high risk or a non-high risk of PTC recurrence based on the expression levels of the first gene set. If the patient is determined to have a non-high risk of PTC recurrence, the non-high risk group may be subclassified based on the level of expression of the second gene set in order to determine whether the patient has a low risk or intermediate risk of PTC recurrence.

Thus, in some embodiments, the step of determining the level of expression of the two or more genes or gene products of the gene signature may comprise determining the level of expression of at least two of: ATG14, MYO3A, ERCC5, SLC43A1, ABCC8, LTK, COPS2, CCNA2, BNIP3, FAM86C1P, GNG4, GCFC2, EEF1A2, TXNL4B, SEPSECS, ZNF215, KIF4A, EZH2, CDCA8, DISP1, SNX29P2, ATP1B1, ZNF620, HIST4H4, CENPL, GATAD1, C2orf88, WWC3, SKA3, HJURP, LOC728613, GTPBP8, RPRM, FBXO4, TICRR, AGFG2, TTK, TAFA2, MTMR14, WDR1, NEK2, RRAGA, EIF2A, and REP15; and the step of determining the patient's risk of PTC recurrence may comprise determining if the patient has a high risk of PTC recurrence. Then, in such embodiments, if the patient is determined not to have a high risk of PTC recurrence, the methods of the present disclosure may further comprise: determining the level of expression of at least two of the genes or gene products of the gene signature; and determining if the patient has an intermediate risk or a low risk of PTC recurrence.

Further, in some embodiments, if the patient is determined to have an intermediate risk of PTC recurrence, the methods of the present disclosure may further comprise determining a subtype of intermediate risk of PTC recurrence. For example, the methods of the present disclosure may further comprise determining the amount of BRAF^{V600E} mutations and/or the amount of RAS mutations in the RNA of the biological sample. The intermediate risk subtype assigned to the patient may indicate the type of treatment most suitable to administer.

The methods of the present disclosure may be applied in a number of ways. For example, in some embodiments, the RNA sample may be isolated and the level of expression of two or more genes or gene products of the gene signature described herein may then be determined. Alternatively, the methods may be applied to a dataset previously collected from an isolated RNA sample. That is, using the previously-collected dataset, the expression levels of two or more genes or gene products of the gene signature described herein may be determined so that the patient may then be classified as having a low risk, intermediate risk, or high risk of PTC recurrence. Such methods may be particularly suitable for computer-based implementation, as will be discussed in greater detail below.

Thus, the methods of the present disclosure involve acquiring data about a new genetic expression pattern, which may also be referred to as a gene signature, for determining the level of risk of recurrence of PCT in a patent. As well, in view of the above, it is clear that the methods of the present disclosure are advantageously capable of being performed entirely in vitro.

For example, the present disclosure also relates to an in vitro method of determining the risk of recurrence of papillary thyroid cancer (PTC) in a patient, the method comprising: isolating an RNA sample from a biological sample of the patient; determining from the RNA sample the level of expression of two or more genes or gene products of a gene signature comprising: ATG14, MYO3A, ERCC5, SLC43A1, ABCC8, LTK, COPS2, CCNA2, BNIP3, FAM86C1P, GNG4, GCFC2, EEF1A2, TXNL4B, SEPSECS, ZNF215, KIF4A, EZH2, CDCA8, DISP1, SNX29P2, ATP1B1, ZNF620, HIST4H4, CENPL, GATAD1, C2orf88, WWC3, SKA3, HJURP, LOC728613, GTPBP8, RPRM, FBXO4, TICRR, AGFG2, TTK, TAFA2, MTMR14, WDR1, NEK2, RRAGA, EIF2A, REP15, NUDT15, LANCL2, NFATC2IP, GTPBP2, KHNYN, CLDN12, DNAH11, ASPHD1, REXO5, HIST2H2BF, C12orf76, MUC21, PGBD5, ABCC6P1, RHBDF1, CHAF1B, MOV10, CAB39L, FN1, DDX19B, BUB1, GPSM2, MSH5, ETV7, SUN1, GRAMD1C, LACTB2, LOC652276, EXOSC10, NUP210, ACOX3, UNC5CL, GNAO1, CGN, ZC3H18, CTSC, MFSD13A, and CCDC183; and determining whether the patient has a low risk, intermediate risk, or high risk of PTC recurrence based on the level of expression of the two or more genes of the gene signature. In such embodiments, the biological sample may be a formalin-fixed paraffin embedded (FFPE) tumor sample, a frozen biopsy tumor sample, or the like.

The present disclosure also relates to methods of treating a patient having papillary thyroid cancer (PTC). In general, the methods of treating involve determining the risk of the recurrence of PTC in the patient, and then administering an appropriate treatment.

Thus, some embodiments of the present disclosure relate to a method of treating a patient having papillary thyroid cancer (PTC), the method comprising: isolating RNA from a biological sample of a tumor of the patient; determining from the RNA the level of expression of two or more genes or gene products of a gene signature comprising: ATG14, MYO3A, ERCC5, SLC43A1, ABCC8, LTK, COPS2, CCNA2, BNIP3, FAM86C1P, GNG4, GCFC2, EEF1A2, TXNL4B, SEPSECS, ZNF215, KIF4A, EZH2, CDCA8, DISP1, SNX29P2, ATP1B1, ZNF620, HIST4H4, CENPL, GATAD1, C2orf88, WWC3, SKA3, HJURP, LOC728613, GTPBP8, RPRM, FBXO4, TICRR, AGFG2, TTK, TAFA2, MTMR14, WDR1, NEK2, RRAGA, EIF2A, REP15, NUDT15, LANCL2, NFATC2IP, GTPBP2, KHNYN, CLDN12, DNAH11, ASPHD1, REXO5, HIST2H2BF, C12orf76, MUC21, PGBD5, ABCC6P1, RHBDF1, CHAF1B, MOV10, CAB39L, FN1, DDX19B, BUB1, GPSM2, MSH5, ETV7, SUN1, GRAMD1C, LACTB2, LOC652276, EXOSC10, NUP210, ACOX3, UNC5CL, GNAO1, CGN, ZC3H18, CTSC, MFSD13A, and CCDC183; determining whether the patient has a low risk, intermediate risk, or high risk of PTC recurrence based on the level of expression of the two or more genes of the gene signature; and administering a treatment to the patient based on the risk of PTC recurrence.

The steps of isolating the RNA from the biological sample, determining the level of expression of the two or more genes or gene products from the gene signature, and determining whether the patient has a low risk, intermediate risk, or high risk of PTC recurrence based on the level of expression of the two or more genes of the gene signature may be performed in the same manners as previously described herein.

In regards to treating the patient based on the risk of PTC recurrence, as previously described herein, different treatments may be appropriate for different levels of risk. For example, for patients determined to have a low risk or intermediate risk of recurrence of PTC, it may be appropriate to administer a treatment that is non-invasive, that has fewer potential side effects, and/or reduced risk of complications. As well, for patients determined to have a high risk of recurrence of PTC, it may be appropriate to administer a more intensive treatment.

In an embodiment, when the patient is determined to have the low-risk or the intermediate-risk of PTC recurrence, the treatment comprises active surveillance and/or a hemithyroidectomy. Active surveillance, as discussed above, involves a series of follow-up appointments and tests to monitor any recurrence of the cancer. The frequency of such appointments and tests may be influenced by the level of risk of recurrence of PTC that the patient is determined to have (e.g. low-risk vs. intermediate-risk). Hemithyroidectomies involve the removal of a portion, for example about half, or less than half or more than half of the thyroid gland. As discussed above, because a hemithyroidectomy removes only a portion of the thyroid gland, a patient may not need life-long replacement of thyroid hormones, as is required for total thyroidectomies. While active surveillance and hemithyroidectomies bear fewer side effects and long-term complications, they may not be sufficient to fully treat more aggressive cases of PTC.

According to a further embodiment of the present disclosure, when a patient is determined to have the high risk of PTC recurrence, the treatment may comprise a total thyroidectomy, adjuvant radioactive iodine (RAI) therapy, administration of one or more inhibitors such as EZH2 inhibitors and one or more immune checkpoint inhibitors, or any combination thereof. Total thyroidectomies are major surgeries that involve the removal of the entire thyroid gland and that bear significant risks and long-term side effects for patients. For example, in addition to life-long replacement of thyroid hormones, the patient may also experience temporary or permanent hypoparathyroidism, or temporary or permanent recurrent laryngeal nerve dysfunction (causing voice changes).

RAI therapy involves administering a radioactive isotope of iodine (I-131) to the patient. The RAI collects in the thyroid gland cells, where the radiation can destroy the thyroid gland or any thyroid tissue remaining after a thyroidectomy as well as any thyroid cancer cells. However, RAI therapy may result in a variety of side effects including nausea and vomiting, ageusia (loss of taste), salivary gland swelling, and pain. As well, RAI therapy may also result in longer-term complications such as recurrent sialoadenitis associated with xerostomia, mouth pain, dental caries, pulmonary fibrosis, nasolacrimal outflow obstruction, and second malignancies. Thus, total thyroidectomies and RAI therapy should only be administered when necessary (for example, in some cases, to patients determined to have a high risk of PTC recurrence).

In the context of the present disclosure, inhibitors are medications that may be used to inhibit one or more biological functions to slow or stop the spread of a cancer. For example, immune checkpoint inhibitors may inhibit immune system checkpoint proteins so that T cells can recognize and attack tumors. EZH2 inhibitors, on the other hand, may inhibit unwanted histone methylation of tumor suppressor genes. In some embodiments of the present disclosure, the inhibitors may be used alone to treat the PTC or in combination with other treatments such as RAI therapy. For example, if a patient is determined to have a high risk or an intermediate risk of PTC recurrence, they may be pretreated with an EZH2 inhibitor and then treated with RAI therapy.

Further, as indicated above, in some embodiments, the intermediate-risk group may be further subclassified into a first intermediate-risk group having high prevalence of BRAF^{V600E} mutations (BRAF_{HIGH}) and a second intermediate risk group enriched with RAS mutations and few BRAF^{V600E} mutations (BRAF_{LOW}). In such embodiments, patients determined to have a BRAF_{HIGH} type intermediate risk of PTC recurrence may be treated with inhibitors such as EZH2 inhibitors and immune checkpoint inhibitors alone or in combination with RAI therapy, while patients determined to have a BRAF_{LOW} type intermediate risk of PTC recurrence may be treated with RAI therapy.

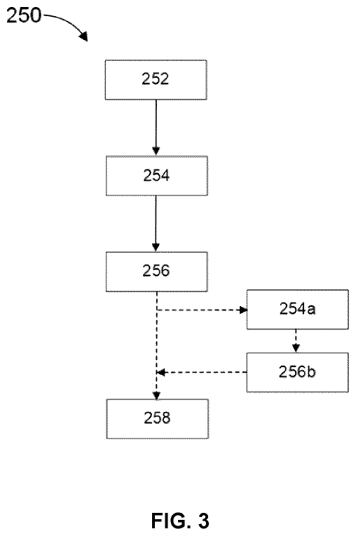

For greater clarity, a flowchart of a method 250 of determining the risk of recurrence of PTC in a patient is shown in FIG. 3. As shown, the method 250 comprises a step 252 of isolating RNA from a biological sample of the patient; a step 254 of determining a level of expression of each of two or more genes of the gene signature of the present disclosure from the RNA; and a step 256 of determining if the patient has a low risk, an intermediate risk, or a high risk of recurrence of PTC based on the level of expression of the two or more genes of the gene signature. Also shown are the optional steps 254a of determining the level of expression of two or more genes of the second gene set (e.g. the gene signature of the present disclosure), as previously described herein, and 256b of determining if the patient has a low risk or an intermediate risk of PTC recurrence. There is also shown the optional step 258 of administering a treatment to the patient based on the determined risk of PTC recurrence.

Some embodiments of the present disclosure relate to the use of a patient's sample and use of the gene signature described herein to provide a prognosis, diagnosis and/or treatment for thyroid cancer.

In some embodiments of the present disclosure, the expression level of the two or more genes or gene products of the gene signature may be determined by analysis of ribonucleic acid (RNA) obtained from a patient's biological sample.

In some embodiments of the present disclosure, the expression level of two or more proteins encoded by the genes contained in the gene signature may be determined by analysis of the applicable proteins from the patient's biological sample.

In some embodiments of the present disclosure, the patient's biological sample may contain cells of a single cell type, multiple cell types or it may be substantially free of cells. The patient's biological sample may be a tissue sample with one or more tissue types therein, a fluid sample with one or more fluid types therein, or a combination of a tissue sample and a fluid sample.

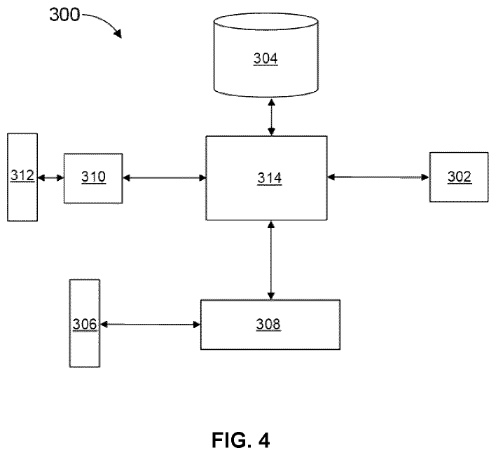

The present disclosure also relates to a system for determining a risk of recurrence of a papillary thyroid cancer (PTC) in a patient. An example of such as system is shown in FIG. 4 and is generally identified using reference numeral 300. As shown, the system 300 of the present disclosure comprises at least one server computer 302, at least one database 304 for storing gene expression information received from a biological sample of a patient 306 by a laboratory 308, and at least one computing device 310 that is accessible by a clinician 312.

The at least one server computer 302, the at least one database 304, the laboratory 308, and the at least one computing device 310, are functionally interconnected by a network 314, such as the Internet, a local area network (LAN), a wide area network (WAN), a metropolitan area network (MAN), or combinations thereof via suitable wired and wireless networking connections.

Each of the at least one server computers 302 executes one or more server programs. The server programs may receive and access the gene expression data determined by the laboratory 308 that is stored on the at least one database 304 to then analyze the expression levels of at least two genes or gene products of the gene signature of the present disclosure. Based on the expression levels of the at least two genes of the gene signature of the present disclosure, the server programs may then determine whether the patient 306 has a high risk, intermediate risk, or low risk of PTC recurrence. The one or more server programs may implement a predictor algorithm or a classifier algorithm to classifying the risk of PTC recurrence of the patient using the expression levels of the at least two genes or gene products of the gene signature of the present disclosure stored in the at least one database 304. The predictor or classifier algorithm may comprise a statistical model such as a regression-based model (e.g. a logistic regression model), a machine learning algorithm (e.g. decision-tree based algorithms such as random forests, Bayes' theorem-based algorithms such as Naïve Bayes classifiers, k-nearest neighbors-based algorithms such as radial basis function networks, support vector machines, and ensemble learning algorithms), or an artificial intelligence (e.g. artificial neural networks), as described above.

Depending on implementation, the server computer 302 may be a server computing device, and/or a general purpose computing device acting as a server computer while also being used by a user.

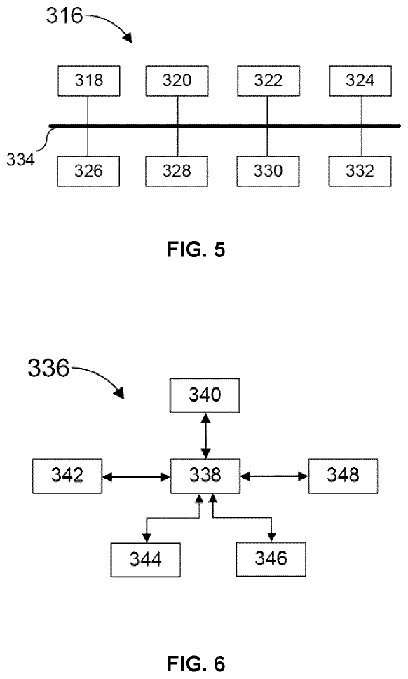

Once a prognosis for the patient 306 is determined by the server programs, the results are communicated to the at least one computing device 310 to be accessed by the clinician 312. The at least one computing device 310 may be a desktop computer, a laptop computer, a tablet, a smartphone, a Personal Digital Assistants (PDAs), or the like. The at least one computing device may have a hardware structure such as a hardware structure 316 shown in FIG. 5.

As shown, the computing device hardware structure 316 comprises a processing structure 318, a controlling structure 320, memory or storage 322, a networking interface 324, coordinate input 326, display output 328, and other input and output modules 330 and 332, all functionally interconnected by a system bus 334.

The processing structure 318 may be one or more single-core or multiple-core computing processors such as INTEL® microprocessors (INTEL is a registered trademark of Intel Corp., Santa Clara, Calif., USA), AMD® microprocessors (AMD is a registered trademark of Advanced Micro Devices Inc., Sunnyvale, Calif., USA), ARM® microprocessors (ARM is a registered trademark of Arm Ltd., Cambridge, UK) manufactured by a variety of manufactures such as Qualcomm of San Diego, Calif., USA, under the ARM® architecture, or the like.

The controlling structure 320 comprises a plurality of controllers such as graphic controllers, input/output chipsets, and the like, for coordinating operations of various hardware components and modules of the at least one computing device 310.

The memory 322 comprises a plurality of memory units accessible by the processing structure 318 and the controlling structure 320 for reading and/or storing data, including input data and data generated by the processing structure 318 and the controlling structure 320. The memory 322 may be volatile and/or non-volatile, non-removable or removable memory such as RAM, ROM, EEPROM, solid-state memory, hard disks, CD, DVD, flash memory, or the like. In use, the memory 322 is generally divided to a plurality of portions for different use purposes. For example, a portion of the memory 322 (denoted as storage memory herein) may be used for long-term data storing, for example, storing files or databases. Another portion of the memory 322 may be used as the system memory for storing data during processing (denoted as working memory herein).

The networking interface 324 comprises one or more networking modules for connecting to other computing devices or networks through the network 314 by using suitable wired or wireless communication technologies such as Ethernet, WI-FI®, (WI-FI is a registered trademark of Wi-Fi Alliance CORPORATION CALIFORNIA, Austin, TEXAS, USA), BLUETOOTH® (BLUETOOTH is a registered trademark of Bluetooth Sig Inc., Kirkland, Wash., USA), ZIGBEE® (ZIGBEE is a registered trademark of ZigBee Alliance Corp., San Ramon, Calif., USA), 3G and 4G wireless mobile telecommunications technologies, and/or the like. In some embodiments, parallel ports, serial ports, USB connections, optical connections, or the like may also be used for connecting other computing devices or networks although they are usually considered as input/output interfaces for connecting input/output devices.

The display output 328 comprises one or more display modules for displaying images, such as monitors, LCD displays, LED displays, projectors, and the like. The display output 328 may be a physically integrated part of the computing device 310 (for example, the display of a laptop computer or tablet), or may be a display device physically separated from but functionally coupled to other components of the computing device 310 (for example, the monitor of a desktop computer).

The coordinate input 326 comprises one or more input modules for one or more users to input coordinate data, such as touch-sensitive screen, touch-sensitive whiteboard, trackball, computer mouse, touch-pad, and/or other human interface devices (HIDs). The coordinate input 326 may be a physically integrated part of the computing device 310 (for example, the touch-pad of a laptop computer or the touch-sensitive screen of a tablet), or may be a display device physically separated from but functionally coupled to other components of the computing device 310 (for example, a computer mouse). The coordinate input 326 in some implementations may be integrated with the display output 328 to form a touch-sensitive screen or touch-sensitive whiteboard.

The hardware structure 316 may also comprise other input modules 330 such as keyboards, microphones, scanners, cameras, and the like. The hardware device 316 may further comprise other output modules 332 such as speakers, printers, and/or the like.

The system bus 334 interconnects various components 318 to 332 enabling them to transmit and receive data and control signals to/from each other.

FIG. 5 shows a simplified software architecture 336 of the computing device 310. The software architecture 336 comprises an operating system 338, one or more application programs 340, logic memory 342, an input interface 344, an output interface 346, and a network interface 348.

The operating system 338 manages various hardware components of the computing device 310 via the input interface 344 and the output interface 346, manages logic memory 342, manages network communications via the network interface 348, and manages and supports the application programs 340 which are executed or run by the processing structure 318 for performing various jobs.

As those skilled in the art appreciate, the operating system 338 may be any suitable operating system such as MICROSOFT® WINDOWS® (MICROSOFT and WINDOWS are registered trademarks of the Microsoft Corp., Redmond, Wash., USA), APPLE® OS X, APPLE® iOS (APPLE is a registered trademark of Apple Inc., Cupertino, Calif., USA), Linux, ANDROID® (ANDROID is a registered trademark of Google Inc., Mountain View, Calif., USA), or the like.

The input interface 344 comprises one or more input-device drivers managed by the operating system 338 for communicating with respective input devices including the coordinate input 326 and other input module 330. The input interface 346 comprises one or more output-device drivers managed by the operating system 338 for communicating with respective output devices including the display output 328 and other output module 332. Input data received from the input devices via the input interface 344 may be sent to one or more application programs 340 for processing. The output generated by the application programs 340 may be sent to respective output devices via the input interface 346.

The logical memory 342 is a logical mapping of the memory or storage 322 for facilitating the application programs 340 to access. In this embodiment, the logical memory 342 comprises a storage memory area that is usually mapped to non-volatile physical memory, such as hard disks, solid state disks, flash drives, and/or the like, for generally long-term storing data therein. The logical memory 342 also comprises a working memory area that is generally mapped to high-speed, and in some implementations volatile, physical memory such as RAM, for the operating system 338 and/or application programs 340 to generally temporarily store data during program execution. For example, an application program 340 may load data from the storage memory area into the working memory area, and may store data generated during its execution into the working memory area. The application program 340 may also store some data into the storage memory area as required or in response to a user's command.

The server computer 302 generally comprises one or more server application programs 340, which provide server-side functions for managing the system 300.

Many obvious variations of the embodiments set out herein will suggest themselves to those skilled in the art in light of the present disclosure. Such obvious variations are within the full intended scope of the appended claims.

### EXAMPLES

### Example 1: Statistical Comparison of the Methods of the Present Disclosure with the American Thyroid Association (ATA) Disease Recurrence Risk Stratification System

The performance of the methods of the present disclosure using the gene signature described herein were compared to that of the ATA system using the procedure outlined below.

Firstly, each individual case within The Cancer Genome Atlas (TCGA) was assigned a risk score by two practicing clinicians. It is noted that tumor stage, based on the American Joint Committee on Cancer (AJCC) staging system, was documented in the TCGA database.

The methods of the present disclosure and the ATA system were evaluated using the cohorts formed from TCGA patients previously described herein—i.e. a first cohort (n=335) and a second cohort (n=167).

Cox proportional hazard (Cox PH) regression analysis was used to evaluate associations of parameters with survival and to evaluate for interactions and additive predictive power.

It was found that there is no significant interaction between classification of risk of recurrence of PTC using the methods of the present disclosure and assigned AJCC stage (p=0.82). That is, the methods of the present disclosure performed independently of current clinical indices.

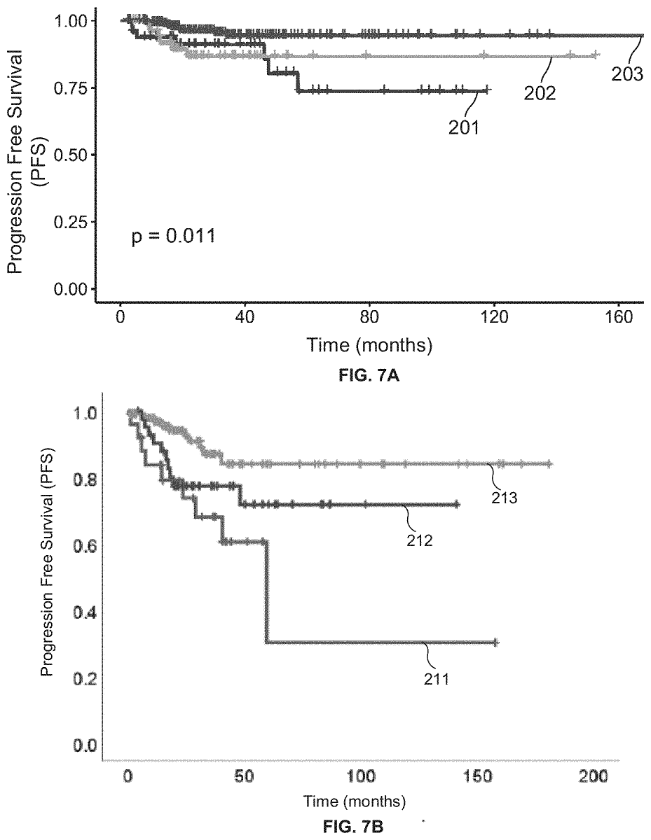

Further, the methods of the present disclosure also outperformed the ATA system in predicting progression free survival (PFS). This is illustrated through a comparison of FIGS. 1A and 7A, which show the predictive performance of the methods of the present disclosure and the ATA system, respectively, based on the first cohort. A comparison of FIGS. 2 and 7B shows the predictive performance of the methods of the present disclosure and the ATA system, respectively, based on the second cohort. In regards to FIG. 7A, it is noted that the line 201 represents the high-risk group, the line 202 represents the intermediate-risk group, and the line 203 represents the low-risk-group. In FIG. 7, the line 211 represents the high-risk group, the line 212 represents the intermediate-risk group, and the line 213 represents the low-risk-group.

As well, Table 2 outlines the concordance scores for the ATA system, the methods of the present disclosure, and a combination thereof.

TABLE 2
Concordance scores for the gene signature of
the present disclosure and the ATA system

| | Prognostic tool | Concordance Score | Wald p-value |
|---|---|---|---|
| Second | Methods of the present | 0.78 | 6 × 10⁻⁵ |
| Cohort | disclosure + ATA | | |
| (n = 167) | Methods of the present | 0.75 | 2 × 10⁻⁵ |
| | disclosure | | |
| | ATA | 0.65 | 0.01 |
| First | Methods of the present | 0.73 | 5 × 10⁻⁵ |
| Cohort | disclosure + ATA | | |
| (n = 335) | Methods of the present | 0.71 | 8 × 10⁻⁴ |
| | disclosure | | |
| | ATA | 0.64 | 0.02 |

The concordance score is an indication of the degree of agreement between two rating techniques. The Wald statistic is an expression of the statistical significance for hypothesis testing of a multiple regression model as compared to a null model of χ²-distribution, which is adjusted for an estimate of the standard error. A low p-value indicates that the model is significant and that the null hypothesis that all variables in a model have regression coefficients equal to zero in the Cox proportional hazard regression model is rejected. Variables with a significant p-value are considered to contribute significantly to the model.

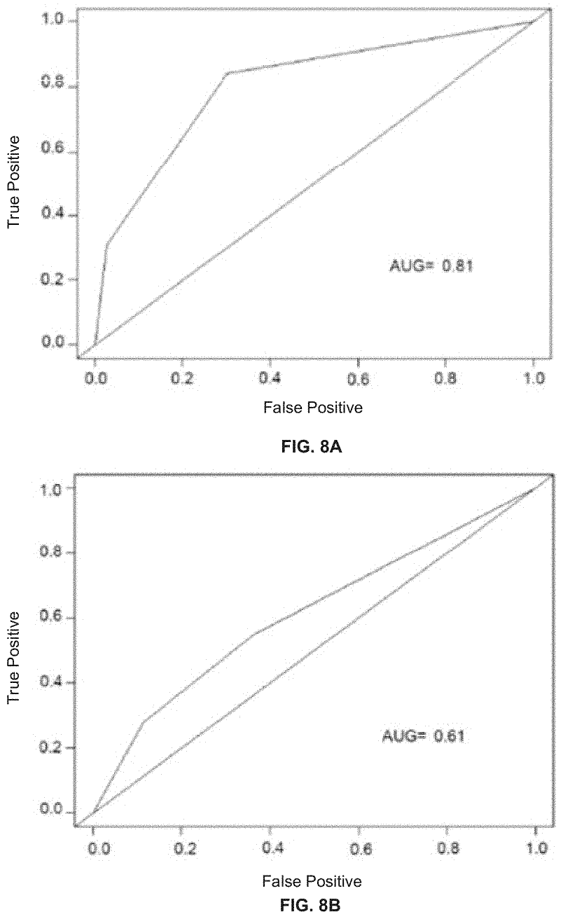

Further, the time-dependent area under the receiver operating characteristic curve (AUROC) for the methods of the present disclosure and the ATA system using the second cohort at a time of four years was also compared. The AUROC was conducted using a nearest neighbour estimation (NNE) method. As shown in FIGS. 8A and 8B, at four years, the methods of the present disclosure have an AUC of 0.81 (FIG. 8A), which outperforms the ATA system having an AUC of 0.61 (FIG. 8B).

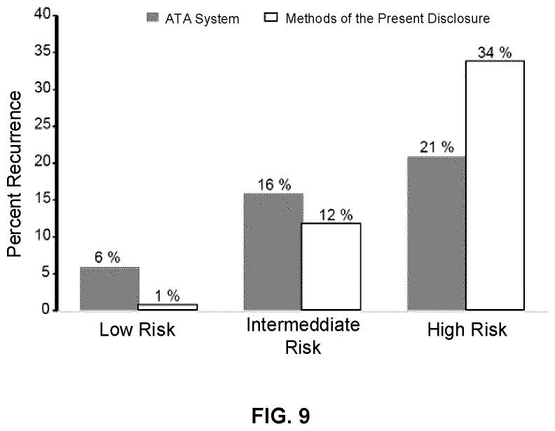

Recurrence risk and the proportions of patients classified as low-risk, intermediate-risk, and high-risk by the methods of the present disclosure and the ATA system were also analysed using the second cohort. The results of this analysis are shown in FIG. 9. Notably, compared to patients classified as low-risk by the ATA system, those classified as having a low-risk of recurrence using the methods of the present disclosure ultimately had a lower recurrence rate. At the same time, the recurrence rate was higher in patients classified as having a high-risk of recurrence using the methods of the present disclosure than those classified as having a high-risk of recurrence by the ATA system. These two observations indicate the methods of the present disclosure may be used to classify risk strata (e.g. low-risk, intermediate-risk, and high-risk) more accurately than the ATA system. In fact, it was found that, in the second cohort, 24% of patients who were classified as having a low-risk of recurrence by the ATA system were reclassified using the methods of the present disclosure as having intermediate- or high-risk risk of recurrence.

### Example 2: Determining the Risk of PTC Recurrence in a Patient Using the Methods of the Present Disclosure

A laboratory collected a tumor sample from a patient via a core biopsy. The laboratory then measured the gene expression levels of the sample using ribonucleic acid sequencing (RNAseq).

Using the gene expression levels determined by the laboratory, the expression levels of the genes of the following first set of genes were analyzed:

ATG14, MYO3A, ERCC5, SLC43A1, ABCC8, LTK, COPS2, CCNA2, BNIP3, FAM86C1P, GNG4, GCFC2, EEF1A2, TXNL4B, SEPSECS, ZNF215, KIF4A, EZH2, CDCA8, DISP1, SNX29P2, ATP1B1, ZNF620, HIST4H4, CENPL, GATAD1, C2orf88, WWC3, SKA3, HJURP, LOC728613, GTPBP8, RPRM, FBXO4, TICRR, AGFG2, TTK, TAFA2, MTMR14, WDR1, NEK2, RRAGA, EIF2A, and REP15.

The patient was determined to have a non-high risk of PTC recurrence. To further classify the patient's risk of PTC recurrence, the expression levels of the genes of the follow second set of genes were analyzed:

ATG14, MYO3A, ERCC5, SLC43A1, ABCC8, LTK, COPS2, CCNA2, BNIP3, FAM86C1P, GNG4, GCFC2, EEF1A2, TXNL4B, SEPSECS, ZNF215, KIF4A, EZH2, CDCA8, DISP1, SNX29P2, ATP1B1, ZNF620, HIST4H4, CENPL, GATAD1, C2orf88, WWC3, SKA3, HJURP, LOC728613, GTPBP8, RPRM, FBXO4, TICRR, AGFG2, TTK, TAFA2, MTMR14, WDR1, NEK2, RRAGA, EIF2A, REP15, NUDT15, LANCL2, NFATC2IP, GTPBP2, KHNYN, CLDN12, DNAH11, ASPHD1, REXO5, HIST2H2BF, C12orf76, MUC21, PGBD5, ABCC6P1, RHBDF1, CHAF1B, MOV10, CAB39L, FN1, DDX19B, BUB1, GPSM2, MSH5, ETV7, SUN1, GRAMD1C, LACTB2, LOC652276, EXOSC10, NUP210, ACOX3, UNC5CL, GNAO1, CGN, ZC3H18, CTSC, MFSD13A, and CCDC183.

The patient was determined to have a low risk of PTC recurrence.

### Definitions

In the present disclosure, all terms referred to in singular form are meant to encompass plural forms of the same. Likewise, all terms referred to in plural form are meant to encompass singular forms of the same. Unless defined otherwise, all technical and scientific terms used herein have the same meaning as commonly understood by one of ordinary skill in the art to which this disclosure pertains.

As used herein, the term “about” refers to an approximately +/−10% variation from a given value. It is to be understood that such a variation is always included in any given value provided herein, whether or not it is specifically referred to.

As used herein, the term “gene expression” refers to the process by which information of a gene is used to produce a functional gene product. Generally, measuring gene expression involves analyzing how the genes are transcribed to produce the functional gene products. Gene expression may be measured using a number of techniques, including reverse-transcription polymerase chain reaction (RT-PCR), complimentary deoxyribonucleic acid (cDNA) microarray, and ribonucleic acid sequencing (RNAseq).

As used herein, the term “gene product” refers to RNA or protein that are products of the transcription and/or translation of a given gene. Examples of gene products include oligonucleotide sequences transcribed from a gene's corresponding DNA sequence such as mature mRNA molecules, gene isoforms, intron sections, exon sections, and protein products formed from translation of the transcribed gene.

As used herein, the term “gene signature” refers to a plurality of genes, as described herein.

As used herein, the expression “high-risk of PTC recurrence” is intended to mean that the risk of recurrence of PTC within 5 years is greater than or equal to about 50%.

As used herein, the expression “intermediate-risk of PTC recurrence” is intended to mean that the risk of recurrence of PTC within 5 years is about 16% to about 49%.

As used herein, the expression “level of expression” refers to determining a level of the genes and gene products thereof, including but not limited to increases, decreases and substantially no change in the detectable levels of the genes of the gene signature and the expression products thereof, including but not limited to: the associated RNA, the associated proteins and/or the genes themselves. Level of expression may also relate to determining a change or substantially no change in the sequence and/or biological activity of such genes and the expression products thereof.

As used herein, “increased expression” refers to an increased abundance of a gene or corresponding gene product as compared to the expression of the given gene or corresponding gene product of a baseline. Increased gene expression may be caused by one or more up-regulation processes within a cell. Further, the “baseline” refers to the abundance of a gene or corresponding gene product measured in a patient having a low risk of PTC recurrence.

As used herein, “decreased expression” refers to a decreased abundance of a gene or corresponding gene product as compared to the expression of the given gene or corresponding gene product of the baseline. Decreased gene expression may be caused by one or more down-regulation processes within a cell.

As used herein, the expression “low-risk of PTC recurrence” refers to the risk of recurrence of PTC within 5 years is less than or equal to about 15%.

As used herein, the term “patient” refers to an animal that may receive, or is receiving, medical treatment, including mammals such as a human patient.

As used herein, the term “prognosis”, “prognostic”, and “prognostication” refer to a forecast of a likely course of action of a disease or ailment, serving to forecast the likely course of action of a disease or ailment, and the action of forecasting a likely course of action of a disease or ailment, respectively.

As used herein, the term “protein” refers to a sequence of amino acids that may be linear or folded into a three dimensional structure such as a secondary, tertiary or quaternary structure, and may contain post-translational elements such as hydrophobic groups.

It should be understood that the compositions and methods are described in terms of “comprising,” “containing,” or “including” various components or steps, the compositions and methods can also “consist essentially of” or “consist of” the various components and steps. Moreover, the indefinite articles “a” or “an,” as used in the claims, are defined herein to mean one or more than one of the element that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

1-30. (canceled)

31. A method of determining a risk of recurrence of a papillary thyroid cancer (PTC) in a patient, the method comprising:
(a) isolating RNA from a biological sample of the patient;
(b) determining a level of expression of each of two or more genes or gene products of a gene signature from the RNA, the gene signature comprising:
ATG14, MYO3A, ERCC5, SLC43A1, ABCC8, LTK, COPS2, CCNA2, BNIP3, FAM86C1P, GNG4, GCFC2, EEF1A2, TXNL4B, SEPSECS, ZNF215, KIF4A, EZH2, CDCA8, DISP1, SNX29P2, ATP1B1, ZNF620, HIST4H4, CENPL, GATAD1, C2orf88, WWC3, SKA3, HJURP, LOC728613, GTPBP8, RPRM, FBXO4, TICRR, AGFG2, TTK, TAFA2, MTMR14, WDR1, NEK2, RRAGA, EIF2A, REP15, NUDT15, LANCL2, NFATC2IP, GTPBP2, KHNYN, CLDN12, DNAH11, ASPHD1, REXO5, HIST2H2BF, C12orf76, MUC21, PGBD5, ABCC6P1, RHBDF1, CHAF1B, MOV10, CAB39L, FN1, DDX19B, BUB1, GPSM2, MSH5, ETV7, SUN1, GRAMD1C, LACTB2, LOC652276, EXOSC10, NUP210, ACOX3, UNC5CL, GNAO1, CGN, ZC3H18, CTSC, MFSD13A, and CCDC183; and
(c) determining if the patient has a low risk, an intermediate risk, or a high risk of recurrence of PTC based on the level of expression of the two or more genes of the gene signature.

32. The method of claim 31, wherein the biological sample is: obtained by macrodissection or microdissection of a tumor; a formalin-fixed paraffin embedded (FFPE) tumor sample; a frozen biopsy tumor sample; a tumor sample that is obtained by fine-needle aspiration; a tumor sample that is obtained by a core biopsy; a tumor sample that is obtained by from a surgical specimen; or any combination thereof.

33. The method of claim 31, wherein the step of determining of the level of gene expression comprises measuring the level of gene expression using a reverse-transcription polymerase chain reaction (RT-PCR), a complimentary deoxyribonucleic acid (cDNA) microarray, or a ribonucleic acid sequencing (RNAseq).

34. The method of claim 31, wherein the step of determining the level of expression of the two or more genes or gene products of the gene signature comprises determining the level of expression of 5 or more genes of the gene signature.

35. The method of claim 34, wherein the step of determining the level of expression of the two or more genes or gene products of the gene signature comprises determining the level of expression of between 20 to 60 genes of the gene signature.

36. The method of claim 31, wherein:
the step of determining the level of expression of the two or more genes of the gene signature comprises determining the level of expression of at least two of:
ATG14, MYO3A, ERCC5, SLC43A1, ABCC8, LTK, COPS2, CCNA2, BNIP3, FAM86C1P, GNG4, GCFC2, EEF1A2, TXNL4B, SEPSECS, ZNF215, KIF4A, EZH2, CDCA8, DISP1, SNX29P2, ATP1B1, ZNF620, HIST4H4, CENPL, GATAD1, C2orf88, WWC3, SKA3, HJURP, LOC728613, GTPBP8, RPRM, FBXO4, TICRR, AGFG2, TTK, TAFA2, MTMR14, WDR1, NEK2, RRAGA, EIF2A, and REP15; and
the step of determining the patient's risk of PTC recurrence comprises determining if the patient has a high risk of PTC recurrence.

37. The method of claim 36, wherein, if the patient is determined not to have a high risk of PTC recurrence, the method further comprises:
determining the level of expression of at least two of the genes or gene products of the gene signature; and
determining if the patient has an intermediate risk or a low risk of PTC recurrence.

38. The method of claim 31, wherein the step of determining the level of expression of the two or more genes or gene products of the gene signature comprises determining the level of expression of at least: ATG14, MYO3A, ERCC5, SLC43A1, ABCC8, LTK, COPS2, CCNA2, BNIP3, FAM86C1P, GNG4, GCFC2, EEF1A2, TXNL4B, SEPSECS, ZNF215, KIF4A, EZH2, CDCA8, DISP1, SNX29P2, ATP1B1, ZNF620, NUDT15, LANCL2, NFATC2IP, GTPBP2, ZNF215, KHNYN, CLDN12, DNAH11, EZH2, ASPHD1, REXO5, HIST2H2BF, C12orf76, MUC21, PGBD5, ABCC6P1, RHBDF1, CHAF1B, MOV10, CAB39L, FN1, DDX19B, and BUB1.

39. The method of claim 31, wherein the step of determining if the patient has a low risk, an intermediate risk, or a high risk of recurrence of PTC recurrence comprises using a statistical model trained using the expression levels of the genes of the gene signature from a plurality of patients in combination with corresponding recurrence data of the plurality of patients.

40. A method of treating a patient having papillary thyroid cancer (PTC), the method comprising:
(a) isolating RNA from a biological sample of the patient;
(b) determining a level of expression of each of two or more genes of a gene signature from the RNA, the gene signature comprising:
ATG14, MYO3A, ERCC5, SLC43A1, ABCC8, LTK, COPS2, CCNA2, BNIP3, FAM86C1P, GNG4, GCFC2, EEF1A2, TXNL4B, SEPSECS, ZNF215, KIF4A, EZH2, CDCA8, DISP1, SNX29P2, ATP1B1, ZNF620, HIST4H4, CENPL, GATAD1, C2orf88, WWC3, SKA3, HJURP, LOC728613, GTPBP8, RPRM, FBXO4, TICRR, AGFG2, TTK, TAFA2, MTMR14, WDR1, NEK2, RRAGA, EIF2A, REP15, NUDT15, LANCL2, NFATC2IP, GTPBP2, KHNYN, CLDN12, DNAH11, ASPHD1, REXO5, HIST2H2BF, C12orf76, MUC21, PGBD5, ABCC6P1, RHBDF1, CHAF1B, MOV10, CAB39L, FN1, DDX19B, BUB1, GPSM2, MSH5, ETV7, SUN1, GRAMD1C, LACTB2, LOC652276, EXOSC10, NUP210, ACOX3, UNC5CL, GNAO1, CGN, ZC3H18, CTSC, MFSD13A, and CCDC183,
(c) determining if the patient has a low risk, an intermediate risk, or a high risk of recurrence of PTC based on the level of expression of the two or more genes of the gene signature; and
(d) administering a treatment to the patient based on the determined level of risk of PTC recurrence.

41. The method of claim 40, wherein the biological sample is: obtained by macrodissection or obtained by microdissection of a tumor; a formalin-fixed paraffin embedded (FFPE) tumor sample; a frozen biopsy tumor sample; a tumor sample that is obtained by fine-needle aspiration; a tumor sample that is obtained by a core biopsy; a tumor sample that is obtained by from a surgical specimen; or any combination thereof.

42. The method of claim 40, wherein the step of determining of the level of gene expression comprises measuring the level of gene expression using a reverse-transcription polymerase chain reaction (RT-PCR), a complimentary deoxyribonucleic acid (cDNA) microarray, or a ribonucleic acid sequencing (RNAseq).

43. The method of claim 40, wherein the step of determining the level of expression of the two or more genes of the gene signature comprises determining the level of expression of 5 or more genes of the gene signature.

44. The method of claim 40, wherein the step of determining the level of expression of the two or more genes of the gene signature comprises determining the level of expression of 20 to 60 genes of the gene signature.

45. The method of claim 40, wherein:
the step of determining the level of expression of the two or more genes of the gene signature comprises determining the level of expression of at least two of:
ATG14, MYO3A, ERCC5, SLC43A1, ABCC8, LTK, COPS2, CCNA2, BNIP3, FAM86C1P, GNG4, GCFC2, EEF1A2, TXNL4B, SEPSECS, ZNF215, KIF4A, EZH2, CDCA8, DISP1, SNX29P2, ATP1B1, ZNF620, HIST4H4, CENPL, GATAD1, C2orf88, WWC3, SKA3, HJURP, LOC728613, GTPBP8, RPRM, FBXO4, TICRR, AGFG2, TTK, TAFA2, MTMR14, WDR1, NEK2, RRAGA, EIF2A, and REP15; and
the step of determining the patient's risk of PTC recurrence comprises determining if the patient has a high risk of PTC recurrence.

46. The method of claim 40, wherein, if the patient is determined not to have a high risk of PTC recurrence, the method further comprises:
determining the level of expression of at least two of the genes of the gene signature; and
determining if the patient has an intermediate risk or a low risk of PTC recurrence.

47. The method of claim 40, wherein the step of determining the level of expression of the two or more genes of the gene signature comprises determining the level of expression of at least: ATG14, MYO3A, ERCC5, SLC43A1, ABCC8, LTK, COPS2, CCNA2, BNIP3, FAM86C1P, GNG4, GCFC2, EEF1A2, TXNL4B, SEPSECS, ZNF215, KIF4A, EZH2, CDCA8, DISP1, SNX29P2, ATP1B1, ZNF620, NUDT15, LANCL2, NFATC2IP, GTPBP2, ZNF215, KHNYN, CLDN12, DNAH11, EZH2, ASPHD1, REXO5, HIST2H2BF, C12orf76, MUC21, PGBD5, ABCC6P1, RHBDF1, CHAF1B, MOV10, CAB39L, FN1, DDX19B, and BUB1.

48. The method of claim 40, wherein if the patient is determined to have a high risk of PTC recurrence, the treatment further comprises performing a total thyroidectomy, administering an adjuvant radioactive iodine (RAI) therapy, administering an immune checkpoint inhibitor, or a combination thereof and wherein, if the patient is determined to have an intermediate risk of PTC recurrence, the treatment comprises performing active surveillance, performing a hemithyroidectomy, administering an adjuvant radioactive iodine (RAI) therapy, or a combination thereof.

49. The method of claim 48, wherein the RAI therapy comprises a pre-treatment with an EZH2 inhibitor.

50. A method of determining a risk of recurrence of a papillary thyroid cancer (PTC) in a patient, the method comprising:
(a) determining a level of expression of each of two or more genes or gene products of a gene signature from the RNA isolated from a biological sample of the patient, the gene signature comprising:
ATG14, MYO3A, ERCC5, SLC43A1, ABCC8, LTK, COPS2, CCNA2, BNIP3, FAM86C1P, GNG4, GCFC2, EEF1A2, TXNL4B, SEPSECS, ZNF215, KIF4A, EZH2, CDCA8, DISP1, SNX29P2, ATP1B1, ZNF620, HIST4H4, CENPL, GATAD1, C2orf88, WWC3, SKA3, HJURP, LOC728613, GTPBP8, RPRM, FBXO4, TICRR, AGFG2, TTK, TAFA2, MTMR14, WDR1, NEK2, RRAGA, EIF2A, REP15, NUDT15, LANCL2, NFATC2IP, GTPBP2, KHNYN, CLDN12, DNAH11, ASPHD1, REXO5, HIST2H2BF, C12orf76, MUC21, PGBD5, ABCC6P1, RHBDF1, CHAF1B, MOV10, CAB39L, FN1, DDX19B, BUB1, GPSM2, MSH5, ETV7, SUN1, GRAMD1C, LACTB2, LOC652276, EXOSC10, NUP210, ACOX3, UNC5CL, GNAO1, CGN, ZC3H18, CTSC, MFSD13A, and CCDC183; and
(b) determining if the patient has a low risk, an intermediate risk, or a high risk of recurrence of PTC based on the level of expression of the two or more genes of the gene signature.
